SEQUENCE LISTING (29 sequences)

SEQ ID NO: 1 - DNA, 24 bp, Bacteriophage fd.
gctgctgagggtgacgatcccgca

SEQ ID NO: 2 - protein, 8 aa, Bacteriophage fd.
AAEGDDPA

SEQ ID NO: 3 - DNA, 83 bp, Artificial
  synthetic
gctgctgagggtggccaacgtggccggtaatgaagcccgcctaatgagcgggcttttttttggcctctggggccgatcccgca

SEQ ID NO: 4 - protein, 9 aa, Artificial
  synthetic
AAEGGQRGR

SEQ ID NO: 5 - protein, 6 aa, Artificial
  synthetic
SGADPA

SEQ ID NO: 6 - DNA, 20 bp, Artificial
  synthetic
gccttcgtagtggcattacg

SEQ ID NO: 7 - DNA, 61 bp, Artificial
  synthetic
agcccgctcattaggcgggcttcattaccggccacgtcggccaccctcagcagcgaaagac

SEQ ID NO: 8 - DNA, 68 bp, Artificial
  synthetic
cggtaatgaagcccgcctaatgagcgggcttttttttggcctctggggccgatcccgcaaaagcggcc

SEQ ID NO: 9 - DNA, 19 bp, Artificial
  synthetic
ggtcagacgattggccttg

SEQ ID NO: 10 - DNA, 42 bp, Artificial
  synthetic
gagctcgctagcgctcgagccaaaaaaaaaggctccaaaagg

SEQ ID NO: 11 - DNA, 20 bp, Artificial
  synthetic
ctagagcagggtccagctaa

SEQ ID NO: 12 - DNA, 12 bp, Artificial
  synthetic
ctggaccctgct

SEQ ID NO: 13 - DNA, 68 bp, Artificial
  synthetic
tcgagctgttgacaattaatcatcggctcgtataatgtgtggaattgtgagcggataacaattgagct

SEQ ID NO: 14 - DNA, 60 bp, Artificial
  synthetic
caattgttatccgctcacaattccacacattatacgagccgatgattaattgtcaacagc

SEQ ID NO: 15 - DNA, 20 bp, Artificial
  synthetic
ccgtgcatctgtcctcgttc

SEQ ID NO: 16 - DNA, 76 bp, Artificial
  synthetic
caggcttaagcatcgacgtcttatcaagacgccttgcttgtaaactttttgaataacttgataccgatagttgcgc

SEQ ID NO: 17 - DNA, 77 bp, Artificial
  synthetic
gtcttgataagacgtcgatgcttaagcctggctagccatcagatctgagtcggccgctgtttaagaaattcacctcg

SEQ ID NO: 18 - DNA, 34 bp, Artificial
  synthetic
gccgtaccgctagcattagaaaaactcatcgagc

SEQ ID NO: 19 - DNA, 75 bp, Artificial
  synthetic
gcttcctgacaggaggccgttttgttttgcagcccacctgagctcccagcttaaggtgtctcaaaatctctgatg

SEQ ID NO: 20 - DNA, 59 bp, Artificial
  synthetic
ggctgagggacgtcgagggcatgcgtacccgataaaagcggcttcctgacaggaggccg

SEQ ID NO: 21 - DNA, 20 bp, Artificial
  synthetic
agcctttcaggtcagaaggg

SEQ ID NO: 22 - DNA, 20 bp, Artificial
  synthetic
attcatgccggagagggtag

SEQ ID NO: 23 - DNA, 22 bp, Artificial
  synthetic
cataacaccccttgtattactg

SEQ ID NO: 24 - DNA, 53 bp, Artificial
  synthetic
gcttacataaacagtaatacaaggggtgttatgaatagttcgacaaagatcgc

SEQ ID NO: 25 - DNA, 37 bp, Artificial
  synthetic
ggatcgagagctagcataactaagcacttgtctcctg

SEQ ID NO: 26 - DNA, 47 bp, Artificial
  synthetic
tgccggcgctcccgctggtttcgctatcctgtcagcacccgggtctg

SEQ ID NO: 27 - DNA, 47 bp, Artificial
  synthetic
acccgggtgctgacaggatagcgaaaccagcgggagcgccggcacgt

SEQ ID NO: 28 - protein, 8 aa, Artificial
  synthetic
APAGFAIL

SEQ ID NO: 29 - DNA, 8233 bp, Bacteriophage fd.
aatagtggactcttgttccaaactggaacaacactcacaactaactcggcctattcttttgatttataaggatttttgtcattttctgcttactggttaaaaaataagctgatttaacaaatatttaacgcgaaatttaacaaaacattaacgtttacaatttaaatatttgcttatacaatcatcctgtttttggggcttttctgattatcaaccggggtacatatgattgacatgctagttttacgattaccgttcatcgattctcttgtttgctccagactttcaggtaatgacctgatagcctttgtagacctctcaaaaatagctaccctctccggcatgaatttatcagctagaacggttgaatatcatattgacggtgatttgactgtctccggcctttctcacccgtttgaatctttgcctactcattactccggcattgcatttaaaatatatgagggttctaaaaatttttatccctgcgttgaaattaaggcttcaccagcaaaagtattacagggtcataatgtttttggtacaaccgatttagctttatgctctgaggctttattgcttaattttgctaactctctgccttgcttgtacgatttattggatgttaacgctactaccattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgaactacagcaccagattcagcaattaagctctaagccatccgcaaaaatgacctcttatcaaaaggagcaattaaaggtactgtctaatcctgacctgttggaatttgcttccggtctggttcgctttgaggctcgaattgaaacgcgatatttgaagtctttcgggcttcctcttaatctttttgatgcaattcgctttgcttctgactataatagacagggtaaagacctgatttttgatttatggtcattctcgttttctgaactgtttaaagcatttgagggggattcaatgaatatttatgacgattccgcagtattggacgctatccagtctaaacattttacaattaccccctctggcaaaacttcctttgcaaaagcctctcgctattttggtttctatcgtcgtctggttaatgagggttatgatagtgttgctcttaccatgcctcgtaattccttttggcgttatgtatctgcattagttgagtgtggtattcctaaatctcaattgatgaatctttccacctgtaataatgttgttccgttagttcgttttattaacgtagatttttcctcccaacgtcctgactggtataatgagccagttcttaaaatcgcataaggtaattcaaaatgattaaagttgaaattaaaccgtctcaagcgcaatttactacccgttctggtgtttctcgtcagggcaagccttattcactgaatgagcagctttgttacgttgatttgggtaatgaatatccggtgcttgtcaagattactctcgacgaaggtcagccagcgtatgcgcctggtctgtacaccgtgcatctgtcctcgttcaaagttggtcagttcggttctcttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctccgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagttattcaaaaagtttacaagcaaggcgtcttgataagacgtcgagggcatgcgtacccgataaaagcggcttcctgacaggaggccgttttgttttgcagcccacctgagctcccagcttaaggtgtctcaaaatctctgatgttacattgcacaagataaaaatatatcatcatgaacaataaaactgtctgcttacataaacagtaatacaaggggtgttatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagatttccctgttttaattttattggctggtggtgggatcgctttacctgcattacagggagtgatgtctatccaaacaaagagtcatgagcaaggtgctttacagggattattggtgagccttaccaatgcaaccggtgttattggcccattactgtttactgttatttataatcattcactaccaatttgggatggctggatttggattattggtttagcgttttactgtattattatcctgctatcgatgaccttcatgttaacccctcaagctcaggggagtaaacaggagacaagtgcttagttatgctagccatcagatctgcactgctcgagctgttgacaattaatcatcggctcgtataatgtgtggaattgtgagcggataacaatttcacacaggaaacagctatgaagaaaagccttgttttgaaggcgagtgtggcggtcgcaacattggtcccaatgttaagctttgcggcggaaggcggccaacgtggccggtaatgaagcccgcctaatgagcgggcttttttttggcctctggggccgaccctgcgaaggcagcattcgatagtttacaggcaagcgcaactgagtacataggctacgcttgggcaatggtcgtcgttatcgtaggagcgaccataggcataaaactgttcaagaagtttacgtctaaagccagttgatagcggccgctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaataaatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatttctgatttgcttgctattggtcgtggtaatgattcctacgacgaaaataaaaacggtttgcttgttcttgatgaatgcggtacttggtttaatacccgttcatggaatgacaaggaaagacagccgattattgattggtttcttcatgctcgtaaattgggatgggatattatttttcttgttcaggatttatctattgttgataaacaggcgcgttctgcattagctgaacacgttgtttattgtcgccgtctggacagaattactttaccctttgtcggcactttatattctcttgttactggctcaaaaatgcctctgcctaaattacatgttggtgttgttaaatatggtgattctcaattaagccctactgttgagcgttggctttatactggtaagaatttatataacgcatatgacactaaacaggctttttccagtaattatgattcaggtgtttattcatatttaaccccttatttatcacacggtcggtatttcaaaccattaaatttaggtcagaagatgaaattaactaaaatatatttgaaaaagttttctcgcgttctttgtcttgcgataggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattccatcacatatattgatttatgtactgtttcaattaaaaaaggtaattcaaatgaaattgttaaatgtaattaattttgttttcttgatgtttgtttcatcatcttcttttgctcaagtaattgaaatgaataattcgcctctgcgcgatttcgtgacttggtattcaaagcaaacaggtgaatctgttattgtctcacctgatgttaaaggtacagtgactgtatattcctctgacgttaagcctgaaaatttacgcaatttctttatctctgttttacgtgctaataattttgatatggttggctcaattccttccataattcagaaatataacccaaatagtcaggattatattgatgaattgccatcatctgatattcaggaatatgatgataattccgctccttctggtggtttctttgttccgcaaaatgataatgttactcaaacatttaaaattaataacgttcgcgcaaaggatttaataagggttgtagaattgtttgttaaatctaatacatctaaatcctcaaatgtattatctgttgatggttctaacttattagtagttagcgcccctaaagatattttagataaccttccgcaatttctttctactgttgatttgccaactgaccagatattgattgaaggattaattttcgaggttcagcaaggtgatgctttagatttttcctttgctgctggctctcagcgcggcactgttgctggtggtgttaatactgaccgtctaacctctgttttatcttctgcgggtggttcgttcggtatttttaacggcgatgttttagggctatcagttcgcgcattaaagactaatagccattcaaaaatattgtctgtgcctcgtattcttacgctttcaggtcagaagggttctatttctgttggccagaatgtcccttttattactggtcgtgtaactggtgaatctgccaatgtaaataatccatttcagacggttgagcgtcaaaatgttggtatttctatgagtgtttttcccgttgcaatggctggcggtaatattgttttagatataaccagtaaggccgatagtttgagttcttctactcaggcaagtgatgttattactaatcaaagaagtattgcgacaacggttaatttgcgtgatggtcagactcttttgctcggtggcctcactgattacaaaaacacttctcaagattctggtgtgccgttcctgtctaaaatccctttaatcggcctcctgtttagctcccgttctgattctaacgaggaaagcacgttgtacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttctccggctttccccgtcaagctctaaatcgggggatccctttagggttccgatttagtgctttacggcacctcgacctccaaaaacttgatttgggtgatggttcacgtagtgggccatcgccctgatagacggtttttcgccctttgacgttggagtccacgttcttt

(19) United States
(12) Patent Application Publication
(10) Pub. No.: US 2005/0112550 A1
(43) Pub. Date: May 26, 2005

(54) Phage display vector, phages encoded thereby, and methods of use thereof

(76) Inventors: Jonathan M. Gershoni, Herzliya (IL); David Enshell-Seliffers, Mosha Avihcail (IL)

Correspondence Address: BROWDY AND NEIMARK, P.L.L.C.;624 NINTH STREET, NW, SUITE 300, WASHINGTON, DC, 20001-5303, US

(21) Appl. No.: 10/474,148

(22) PCT Filed: Apr. 5, 2002
(86) PCT No.: PCT/US02/10720

Related U.S. Application Data
(60) Provisional application No. 60/281,401, filed on Apr. 5, 2001.

(51) Int. Cl. C12Q 1/70; C07H 21/04; C12N 7/00; C12N 15/74; C12N 1/21; C07K 14/005

(52) U.S. Cl. 435/5; 435/440; 435/69.3; 435/235.1; 530/350; 435/252.3; 536/23.72; 435/472

(57) ABSTRACT

Filamentous bacteriophages are particularly efficient for the expression and display of combinatorial random peptides. Two phage proteins are often employed for peptide display: the infectivity protein, pIII and the major coat protein, pVIII. The use of pVIII typically requires the expression of two pVIII genes: the wild type and the recombinant pVIII genes to generate mosaic phages. “Type 88” vectors contain two pVIII genes in one phage genome. A novel “type 88” expression vector has been rationally designed and constructed, which can be used to express recombinant peptides as pVIII chimeric proteins in mosaic bacteriophages. This vector is not only genetically stable but also of high copy number and produces high titers of recombinant phages.

### FIELD OF THE INVENTION

The present invention is related to a phage display vector encoding a filamentous phage which includes DNA encoding a polypeptide of interest and, more particularly, to such a vector in which a recombinant sequence including a multiple cloning site is inserted between the wild type pVIII and pIII genes. The invention further relates to the phages encoded by such DNA, phage display libraries made of such phages and methods of use thereof.

### BACKGROUND OF THE INVENTION

Combinatorial phage display peptide libraries provide an effective means to study protein:protein interactions. This technology relies on the production of very large collections of random peptides associated with their corresponding genetic blueprints (Scott et al, 1990; Dower, 1992; Lane et al, 1993; Cortese et al, 1994; Cortese et al, 1995; Cortese et al, 1996). Presentation of the random peptides is often accomplished by constructing chimeric proteins expressed on the outer surface of filamentous bacteriophages such as M13, fd and f1. This presentation makes the repertoires amenable to binding assays and specialized screening schemes (referred to as biopanning (Parmley et al, 1988)) leading to the affinity isolation and identification of peptides with desired binding properties. In this way peptides that bind to receptors (Koivunen et al, 1995; Wrighton et al, 1996; Sparks et all, 1994; Rasqualini et al, 1996), enzymes (Matthews et al, 1993; Schmitz et al, 1996) or antibodies (Scott et al, 1990; Cwirla et al, 1990; Felici et al, 1991; Luzzago et al, 1993; Hoess et al, 1993; Bonnycastle et al, 1996) have been efficiently selected.

Filamentous bacteriophages are nonlytic, male specific bacteriophages that infect Escherichia coli cells carrying an F-episome (for review, see Model et al, 1988). Filamentous phage particles appear as thin tubular structures 900 nm long and 10 nm thick containing a circular single stranded DNA genome (the +strand). The life cycle of the phage entails binding of the phage to the F-pilus of the bacterium followed by entry of the single stranded DNA genome into the host. The circular single stranded DNA is recognized by the host replication machinery and the synthesis of the complementary second DNA strand is initiated at the phage ori(−) structure. The double stranded DNA replicating form is the template for the synthesis of single-stranded DNA circular phage genomes, initiating at the ori(+) structure. These are ultimately packaged into virions and the phage particles are extruded from the bacterium without causing lysis or apparent damage to the host.

Peptide display systems have exploited two structural proteins of the phage; pIII protein and pVIII protein. The pIII protein exists in 5 copies per phage and is found exclusively at one tip of the virion (Goldsmith et al, 1977). The N-terminal domain of the pIII protein forms a knob-like structure that is required for the infectivity process (Gray et al, 1981). It enables the adsorption of the phage to the tip of the F-pilus and subsequently the penetration and translocation of the single stranded phage DNA into the bacterial host cell (Holliger et al, 1997). The pIII protein can tolerate extensive modifications and thus has been used to express peptides at its N-terminus. The foreign peptides have been up to 65 amino acid residues long (Bluthner et al, 1996; Kay et al, 1993) and in some instances even as large as full-length proteins (McCafferty et al, 1990; McCafferty et al, 1992) without markedly affecting pIII function.

The cylindrical protein envelope surrounding the single stranded phage DNA is composed of 2700 copies of the major coat protein, pVIII, an α-helical subunit which consists of 50 amino acid residues. The pVIII proteins themselves are arranged in a helical pattern, with the α-helix of the protein oriented at a shallow angle to the long axis of the virion (Marvin et al, 1994). The primary structure of this protein contains three separate domains: (1) the N-terminal part, enriched with acidic amino acids and exposed to the outside environment; (2) a central hydrophobic domain responsible for: (i) subunit:subunit interactions in the phage particle and (ii) transmembrane functions in the host cell; and (3) the third domain containing basic amino acids, clustered at the C-terminus, which is buried in the interior of the phage and is associated with the phage-DNA. pVIII is synthesized as a precoat protein containing a 23 amino acid leader-peptide, which is cleaved upon translocation across the inner membrane of the bacterium to yield the mature 50-residue transmembrane protein (Sugimoto et al, 1977). Use of pVIII as a display scaffold is hindered by the fact that it can tolerate the addition of peptides no longer than 6 residues at its N-terminus (Greenwood et al, 1991; Iannolo et al, 1995). Larger inserts interfere with phage assembly. Introduction of larger peptides, however, is possible in systems where mosaic phages are produced by in vivo mixing the recombinant, peptide-containing, pVIII proteins with wild type pVIII (Felici et al, 1991; Greenwood et al, 1991; Willis et al, 1993). This enables the incorporation of the chimeric pVIII proteins at low density (tens to hundreds of copies per particle) on the phage surface interspersed with wild type coat proteins during the assembly of phage particles. Two systems have been used that enable the generation of mosaic phages; the “type 8+8” and “type 88” systems as designated by Smith (Smith, 1993).

The “type 8+8” system is based on having the two pVIII genes situated separately in two different genetic units (Felici et al, 1991; Greenwood et al, 1991; Willis et al, 1993). The recombinant pVIII gene is located on a phagemid, a plasmid that contains, in addition to its own origin of replication, the phage origins of replication and packaging signal. The wild type pVIII protein is supplied by superinfecting phagemid-harboring bacteria with a helper phage. In addition, the helper phage provides the phage replication and assembly machinery that package both the phagemid and the helper genomes into virions. Therefore, two types of particles are secreted by such bacteria, helper and phagemid, both of which incorporate a mixture of recombinant and wild type pVIII proteins.

The “type 88” system benefits by containing the two pVIII genes in one and the same infectious phage genome. Thus, this obviates the need for a helper phage and superinfection. Furthermore, only one type of mosaic phage is produced. The question arises, however, where one should introduce the second pVIII gene within the filamentous phage genome for efficient expression and genetic stability.

The phage genome encodes 10 proteins (pI through pX) all of which are essential for production of infectious progeny (Felici et al, 1991). The genes for the proteins are organized in two tightly packed transcriptional units separated by two non-coding regions (Van Wezenbeek et al, 1980). One non-coding region, called the “intergenic region” (defined as situated between the pIV and pII genes) contains the (+) and the (−) origins of DNA replication and the packaging signal of the phage, enabling the initiation of capsid formation. Parts of this intergenic region are dispensable (Kim et al, 1981; Dotto et al, 1984). Moreover, this region has been found to be able to tolerate the insertion of foreign DNAs at several sites (Messing, 1983; Moses et al, 1980; Zacher et al, 1980). The second non-coding region of the phage is located between the pVIII and pIII genes, and has also been used to incorporate foreign recombinant genes as was illustrated by Pluckthun (Krebber et al, 1995).

Regardless as to where a second pVIII gene is to be introduced, a major point for concern is the genetic stability of the ultimate vector and its derivatives.

### SUMMARY OF THE INVENTION

The present invention is based on a critical examination of the attributes of the two non-coding regions of the fd filamentous phage as potential sites for insertion of a second recombinant pVIII gene and its genetic stability, resulting in the design and construction of an efficient “type 88” phage display expression system.

The phage display expression system of the present invention includes a vector which is the DNA sequence of a filamentous phage into which a second pVIII gene, as well as DNA encoding a peptide of interest, are placed between the wild type pVIII and pIII genes. This allows production of type 88 phages displaying a peptide of interest with genetic stability and high copy number.

The DNA encoding the second pVIII gene preferably uses alternative codons for encoding the amino acid residues of the pVIII protein. The native pVIII DNA sequence is separated from the recombinant pVIII sequence by a region encoding the wild type pVIII C-terminal domain and a terminator. The region encoding the wild type pVIII C-terminal domain is preferably designed to use alternative codons which are other than the native codons for encoding the same amino acids. In this way homologous recombination, slippage mechanisms and other genetic instabilities may be avoided.

Preferably, the terminator at the end of the native pVIII DNA sequence is an HP terminator which is not native to the filamentous phage DNA.

In a preferred embodiment of the present invention, a positive selection marker, such as the tetracycline or kanamycin resistant genes, is inserted between the native and recombinant pVIII genes. Preferably, a unidirectional promoter is substituted for the native bidirectional tetracycline resistant gene promoter.

In preferred embodiments of the present invention, the native intergenic space between pIV and pII is maintained.

The present invention further relates to the filamentous phages encoded by the DNA discussed above. Such phages may be of any type, such as fd, M13 and f1, although fd is preferred. The peptide designed to be displayed by the phage of the present invention can be any peptide which is desired to be presented, such as a specific antigen. Indeed, any protein or peptide can be displayed on the phage of the present invention.

The peptide displayed on the phage may be an epitope of an antigen, which phage can be used therapeutically as a vaccine. Furthermore, the peptide displayed on the phage may be a single-chain antibody, which phage can be used for passive immunization. Both of these embodiments are described in WO 01/18169.

Alternatively, one can create a library of phages into which are incorporated, at the same site, each of a set of oligonucleotides that encode all possible random peptides of a given length, or a large subset of that set. Such a large subset should preferably represent a fraction of the full set with the theoretical complexity of the full set of random polypeptides of a given length.

Libraries of phages can also be prepared which incorporate all, or a large subset of all, of the overlapping oligonucleotides that represent all of the overlapping peptides of a given antigen and, thus, create a phage display pepscan. Scrambled pepscans (PCT application no. WO 98/20169) and phage display two hybrid systems (PCT application no. WO 98/20159) may also be prepared by this technique.

The phage display libraries of the present invention can be used in screening for molecules which bind to a particular displayed peptide of interest or in screening the peptides displayed on a library of phages to see which bind to a specific molecule of interest, all as is well known with respect to prior art phage display libraries. Once a molecule or peptide of interest is identified by means of the screen using the phages or phage display libraries of the present invention, the peptide or molecule so identified may be produced in a conventional manner.

### BRIEF DESCRIPTION OF THE DRAWINGS

FIGS. 1A and 1B are a comparison between fd (FIG. 1A) and fd-tet (FIG. 1B). Opened arrows indicate the phage-transcriptional units. The darkened segment in FIG. 1B indicates the tet fragment introduced into the BamH I site of fd at position 6272 (bold in FIG. 1A) in the intergenic region (IG).

FIGS. 2A-2B show a deletion of 2.8 kb in fd-tet. FIG. 2A is an uncut double-stranded DNA of fd-tet isolated from K802 (lane 1) or K91KAN (lane 2) and run on 0.8% agarose gel. Uncut double-stranded DNA of wild type fd was used for comparison (lane 3). M: DNA ladder mix (numbers equal base pairs). FIG. 2B shows Msc I digests of fd-tet derived from K802 (lane 1) or K91KAN (lane 2) which were run on 0.8% agarose gel. Msc I digest of fd was used for comparison (lane 3). M: λ Hind III digest (numbers equal base pairs). Note that the lower band in lane 2 in FIG. 2B, corresponding to the deleted product, is at least as intense as the upper band corresponding to the linear full-length fd-tet .

FIG. 3 shows the genetic instability of the inserted tet fragment. SnaB I/Ava I digests of fd-tet derived from K802 (lane 1) or K91KAN (lane 2). SnaB I/Ava I digest of fd was used for comparison (lane 3). BspH I/Hind III digests of fd-tet derived from K802 (lane 4) or K91KAN (lane 5:). BspH I/Hind III digest of fd DNA was used for comparison (lane 6). The arrow indicates the deleted products (lane 2 and 5) corresponding in size to the linear full-length fd (lane 3 and 6). M: DNA ladder mix. Agarose gel concentration was 0.8%.

FIGS. 4A-4B show that the deletion is not a precise reversion to fd. FIG. 4A is BamH I digests of fd-tet DNA derived from K802 (lane 1) or K91KAN (and 2). BamH I digest of fd was used for comparison (lane 3). Note that the pattern obtained in lane 2 is identical to the Msc I digest in FIG. 2B, lane 2. M: DNA ladder mix. Agarose gel concentration was 0.8%. FIG. 4B is BstY I digests of fd-tet derived from K802 (lane 1) or K91KAN (lane 2) run on 0.8% agarose gel. BstY I digest of fd was used for comparison (lane 3). M: DNA ladder mix. Note the relative drop in intensity for the lowest band in FIG. 4B, lane 2.

FIG. 5 shows the genome of the fd-tet phage. Genes (darkened segments), promoters (bent arrows), terminators (stem and loop), and selected restriction sites relevant to this study are shown. Strong promoters and terminators are in black. Weak promoters and terminators are in gray. The wild type fd genes (pI through pX) are packed in two transcriptional units. The inserted tet fragment contains two genes; tetA encoding for the tetracycline resistance protein and tetR encoding for the repressor protein regulating the expression of the tetA gene. The overlapping promoter/terminator, between pVIII and pIII genes, are inseparable. Moreover, the −35 box of this promoter is situated in the C-terminal coding region of pVIII gene (see text). Note the bi-directional promoter driving tetR and teta transcription.

FIGS. 6A-6C show the construction of two tandem pVIII genes. FIG. 6A is a detailed comparison between the wild type pVIII gene (the contiguous central portion being SEQ ID NO:1) and the recombinant pVIII gene (the contiguous central portion being SEQ ID NO:3), designated pVIIISTS. The amino acid sequence shown to be encoded by SEQ ID NO:1 is SEQ ID NO:2. The amino acid sequences shown to be encoded by SEQ ID NO:3 are SEQ ID NOs:4 and 5. In the pVIII gene, the GAC codon for Asp at position 4 of the wild type pVIII is deleted and replaced by a sequence of 62 bp containing two stop codons and trpA transcription terminator flanked by two Sfi I sites (“STS” insert) (nucleotides 13-74 of SEQ ID NO:3). FIG. 6B is the pGEM-t(p8STS) construct. The fragment containing the pVIIISTS gene, preceded by a SnaB I restriction site and followed by downstream pIII gene, was introduced into the pGEM-T vector. Black segments are sequences corresponding to the pGEM-T vector. The 62 bp “STS” insert is indicated. FIG. 6C is the pGEM-t(pB8STS) construct. This construct contains the wild type pVIII and the modified pVIIISTS genes arranged as tandem repeats. The fragment containing the wild type pVIII gene starting from the SnaB I site and ending just beyond the overlapping promoter/terminator was introduced into the SnaB I site of pGEM-T(p8STS) thus destroying the former site while concomitantly introducing a new SnaB I site upstream to the wild type pVIII gene. Furthermore, additional unique sites were introduced between the two pVIII genes.

FIG. 7 is the genome of ftac88 vector. Most of the tetR gene was exchanged for a linker containing the Ava II site, thus eliminating the second SnaB I site previously situated in this region (for comparison see FIG. 5). The modified pVIIISTS gene is situated between the duplicated overlapping promoter/terminator structures, without disrupting the two-phage transcriptional units. Note the pVIIISTS gene is under the control of a tac promoter (Ptac).

FIG. 8 shows the binding of mAb GV4H3 to phages expressing the GV4H3 epitope. Equal amounts of phages were applied in duplicates to a nitrocellulose membrane filter and reacted with the GV4H3 mAb. The GV4H3 epitope displaying phages, ftac88(4H3) and fth1(4H3), showed strong signals. Phage ftac88 and fth1 were used as negative controls.

FIG. 9 shows the deletion of the recombinant pVIII gene in ftac88(4H3). SnaB I/BamH I digests of ftac88 derived from DH5α (lane 1) or DH5αF′ (lane 2) were run on 0.8% agarose gel. SnaB I/BamH I digests of ftac88 (4H3) isolated from DH5α (lane 3) or DH5αF′ (lane 4) are also shown. M: DNA ladder mix. The upper arrow indicates the tet insert deleted product and the lower arrow indicates the recombinant pVIII gene deleted fragment.

FIG. 10 shows the fth1 vector (SEQ ID NO:29). The general scheme of the fth1 vector is shown in scale illustrating the phage genes (opened segments), the reconstituted intergenic region (IG) and the genes introduced between the wild type pVIII and pIII genes (gray segments). Below, detail of the pIX to the middle of pIII is provided (not in scale). The darkened segments represent the modified wild type pVIII 3′-end and the −35 box of the pIII promoter (see also FIG. 11). For details see text.

FIGS. 11A-11C show the intermediate vectors constructed for the formation of fth1 vector. FIG. 11A is a detail of the segment of fd-tet containing the pIX, pVIII, and pIII genes. The segment in pVIII indicating the −35 box of the pIII promoter is shown in black situated in the 3′ region of the wild type pVIII open reading frame. FIG. 11B is the intermediate vector-1 (IV-1). The −35 box of the pIII promoter was separated from the wild type pVIII gene by inserting a small segment reconstituting the 3′ region and introducing multiple cloning sites. FIG. 11C is IV-4. A novel HP terminator (t_{HP}) is situated downstream to the wild type pVIII gene terminating its transcription followed by the tetracycline resistance gene (tetA) under the control of the kan promoter (Pₖₐₙ).

FIG. 12 shows the genetic stability of fth1. BstN I digests of fth1 derived from DH5α (lane 2) or DH5αF′ (lane 3). BstN I digests of fth1(4H3) derived from DH5α (lane 4) or DH5αF′ (lane 5). BstN I digest of fd was used for comparison (lane 1). M: DNA ladder mix. Agarose gel concentration was 0.8%.

### DETAILED DESCRIPTION OF THE INVENTION

Type 88 display systems are in essence a phage genome that contains two protein VIII genes. One of the genes is the wild type pVIII, whereas the second is modified so as to enable expression of a peptide (random or pre-defined peptide, or even a single-chain antibody (scFv)). The problem with known type 88 phage display systems is genetic instability. After a number of generations, the phages tend to stop production of the inserted polypeptide of interest and no longer display such polypeptide. The novel constructs of the present invention solve this problem. Furthermore, in prior art systems, in which the selectable marker has been introduced into the intergenic region, some of the functions of the replication of the phage are obstructed, and the actual amount of phage DNA being produced in a liter of culture may be no more than 20-50 μg of DNA. However, with the construct of the present invention, high copy numbers may be obtained. Thus, as much as 500-1000 μg of DNA can be readily purified from a liter of culture.

In the vector of the present invention, the sequence of a filamentous phage is manipulated so as to insert a second pVIII gene and the gene encoding the polypeptide of interest between the wild type pVIII and pIII genes. In a preferred embodiment, the intergenic region between pII and pIV is not modified and retains the native sequence, thus allowing for the high copy number to be obtained. Any positive selection marker may also be introduced between the wild type pVIII and pIII genes.

In the wild type filamentous phage DNA, the −35 box necessary for pIII expression is positioned to overlap with the C-terminal coding region of the pVIII protein. Thus, the −35 regulatory sequence is upstream of the C-terminal coding region and terminator of the pVIII protein. So as not to disrupt the expression of the pIII protein, the vector of the present invention preferably maintains the genuine C-terminal/−35 box at the 5′ end of the pIII gene. A new C-terminal region for the native pVIII gene is introduced upstream thereof. The second pVIII region and the gene encoding the polypeptide of interest is introduced between the newly-introduced terminator for the native pVIII gene and the C-terminal/−35 box 5′ to the pIII gene.

In order to prevent possible homologous recombination, slippage mechanisms, or other genetic instabilities, it is preferable that the DNA encoding the second pVIII gene use alternative codons which are other than the native codons for encoding amino acid residues of the pVIII protein. Enough alternative codons should be inserted therein so as to avoid stretches longer than 30 bases of the native sequence, preferably no longer than 20 such bases. Similarly, the region encoding the wild type pVIII C-terminal domain should also use alternative codons in sufficient quantity to avoid stretches longer than 30 native bases, and preferably to avoid stretches longer than 20 such bases.

The positive selection marker is preferably inserted between the native and recombinant pVIII genes.

The present invention further comprehends a vector in which the second pVIII gene is introduced just downstream to the −35 promotor of the pIII gene (and thus driven by the pIII promotor and not the tac promotor of fth1 or ftac88 as described). In such a situation, due to the presence of the transcription terminator in the STS stuffer of the recombinant pVIII, phages that do not incorporate an insert for the polypeptide of interest will not be produced at all. This is due to the fact that the terminator prevents the expression of the pIII, pVI and pI proteins. The pI protein is essential for phage assembly. In the situation where an insert is cloned in place of the stuffer, the recombinant phage is made. If such an insert is not incorporated, then transcription is terminated and no phage is produced.

While the fd filamentous phage is used in the present examples and is the preferred phage sequence for use in the present invention, it should be understood that all filamentous phages are very similar and have the same gene organization (Model et al, 1988). Thus, the principles of the present invention can be applied to any of the filamentous phages, such as M13, f1 and others.

The native pVIII DNA sequence is preferably separated from the second pVIII DNA sequence by a terminator which is not native to the filamentous phage DNA, preferably the HP terminator.

In the preferred embodiment of the present invention, the native, unique, overlapping pVIII terminator/pIII promoter is maintained at its native position at the N-terminal portion of the pIII gene. This includes the pVIII C-terminal region/pIII −35 box. However, to further avoid tandem repeats, the pVIII C-terminal region, which is present at this point, is preferably designed with alternative codon usage to avoid stretches of longer than 30 native bases, and preferably to avoid stretches longer than 20 native bases.

While the DNA molecule of the present invention is described as having a recombinant DNA sequence encoding the pVIII protein and DNA encoding a polypeptide of interest other than pVIII which is inserted between the wild type pVIII and pIII genes, this language encompasses the presence of DNA encoding a peptide of interest within the sequence encoding the pVIII protein. Preferably, the DNA sequence encoding the peptide of interest is present at the N-terminal region of the pVIII gene. The peptide of interest and the pVIII gene will be expressed as a fusion protein in which the peptide of interest is expressed on the surface of the filamentous phage.

While the selectable marker disclosed herein is the tetracycline resistance gene, those of ordinary skill in the art will understand that any selectable marker can be used for this purpose, such as the kanamycin resistance gene. Furthermore, as the native promoter for the tetracycline resistance gene is bidirectional, in a preferred embodiment of the present invention, this promoter is replaced by a unidirectional promoter, such as the kanamycin promoter.

The present invention also comprehends a vector which may be used as an intermediate for producing a vector containing the DNA encoding the peptide of interest. In this intermediate vector, the DNA of the phage has inserted therein a recombinant DNA sequence that includes a multiple cloning site. The multiple cloning site has a series of restriction sites that do not otherwise appear in the DNA of the phage into which the recombinant DNA has been inserted. The recombinant DNA sequence is designed and inserted such that the multiple cloning site is placed between a terminator for the wild type pVIII gene and an initiator for the wild type pII gene. The multiple cloning site may then be used to readily insert DNA sequences encoding the foreign peptide of interest to be displayed on the phage. A positive selection marker, as has been discussed elsewhere in the present specification, may be present in the multiple cloning site in order to simplify insertion only of the DNA encoding the peptide of interest so as to arrive at the final product. The second pVIII gene may also be present in the multiple cloning site.

Instead of a second pVIII gene, any recombinant bacteriophage gene can be present in the multiple cloning site. For example, it would be useful to produce a system in which two pIII genes are present. This would allow the production of both wild type and recombinant pIII genes in the same phage. It is expected that, by modifying the efficiency of expression, one can create a situation in which only one recombinant pIII protein is expressed per phage. The normal situation is that five pIII proteins are present per phage. When all are recombinant, then the binding is affected by the multivalency of the five copies which leads to enhanced avidity of binding and can be misleading if one wants to be precise in evaluating real affinity (as opposed to avidity which benefits from multiple binding reactions). Similarly, any of the native proteins (pI through pX) can be duplicated in the multiple cloning site for whatever reason.

The recombinant DNA sequence, which is inserted into the DNA of the filamentous phage, is designed and inserted such that the multiple cloning site is placed between a terminator for the wild type pVIII gene and an initiator for the wild type pIII gene. As discussed hereinabove, preferably the wild type C-terminal/−35 box is retained at the 5′ end of the pIII gene, and a new C-terminal region for the native pVIII gene is introduced upstream of the multiple cloning site.

Preferably, in the intermediate vector, in the region between flanking restriction enzyme sites, a trpA transcription terminator is disposed, although any random combination of bases can appear between these flanking restriction enzyme sites. When this intermediate is to be used for production of the vector to be used to make a phage displaying the peptide of interest, DNA encoding the peptide of interest is merely substituted for the DNA of the intermediate vector between the two flanking unique restriction enzyme sites.

Since the recombinant DNA sequence inserted into the phage in order to create the multiple cloning site must include a region homologous to the N-terminal portion of the pIII gene, it is possible in this same insert to include a DNA sequence encoding another foreign peptide incorporated into the pIII gene at the N-terminal region thereof. This may be used, for example, to produce a phage that displays a library of random peptides on either pIII or pVIII and a marker on the other, such as green fluorescence protein. This would make the selection of the bound phage easier or amenable to high throughput screening.

Also encompassed by the present invention is the recombinant DNA construct which is produced and used for insertion of the multiple cloning sites, into the phage DNA so as to appear between the pVIII and pIII genes. This construct is designed so as to replace the wild type sequence between restriction sites which are already present in the phage, such as the SnaBI in the pIX gene and the BamHI in the pIII gene. The ends of the construct are designed so as to be compatible with the restriction enzyme cleavage sites left after removal of the wild type sequence such that efficient ligation of the construct into the vector may be obtained, all as is well known to those of ordinary skill in the art.

The novel DNA vectors of the present invention and the novel type 88 filamentous phages encoded thereby may be used in the same manner that known peptide display phages have been known to be used in the prior art, except that they are genetically stable, have high copy number, and produce high titres of phages. The vector can be used to express any protein or peptide. In the situation in which a discrete sequence of peptide is known which is desired to be expressed, then such a known peptide can readily be incorporated into the vector so as to produce high titres of phage displaying the desired discrete amino acid sequence.

An example of the use of a discrete sequence of peptide displayed on a phage is represented by the disclosure of PCT application no. WO 01/18169, the entire contents of which are hereby incorporated herein by reference. This publication discloses the utility as a vaccine of phages displaying a particular antigenic epitope so as to raise antibodies against that epitope. Also disclosed therein are phages displaying single-chain antibodies which can be directly used for passive immunization. Thus, phages displaying such antigens or single-chain antibodies for use in the particular applications of WO 01/18169, or any related application, are considered to be comprehended by the present invention when the antigen or single-chain antibody is displayed on a phage in accordance with the present invention.

Additionally, as is known in the art, libraries can be prepared that incorporate into the same site of the vector a set of oligonucleotides that code all possible random peptides of a given length, or a large subset of that set, as is known in the art. Such a large subset should preferably represent a fraction of the full set with the theoretical complexity of the full set of random polypeptides of a given length. Such phage display libraries can be used to screen for amino acid combinations which will bind to a given target molecule. Once a phage which binds to the desired target molecule is found, the peptide insert in that phage can be determined and molecules containing that peptide can be produced. Such molecule would be expected to bind the target molecule. The binding of the target molecule to its native receptor or ligand may be disrupted. This is useful when it is desired to prevent the signaling which occurs when the target molecule is bound to its native ligand or receptor, all as is well known in the art of phage display libraries.

Furthermore, libraries can be prepared which incorporate the set of all of the overlapping oligonucleotides that represent all the overlapping peptides of a given antigen, or a large subset thereof, and, thus, create a phage display pepscan. A scrambled pepscan, such as the combinatorial scrambled vaccines described in PCT application no. WO 98/20169, the entire contents of which are hereby incorporated herein by reference, and the phage display two hybrid systems, such as are described in PCT application no. WO 98/20159, the entire contents of which are hereby incorporated by reference, may also be prepared using the novel phages of the present invention.

Any of such phage display libraries can be used in screen assays as is known in the art. In any such screening assay, the peptide that is found can then be produced and used for its intended purpose, again, as is well known in the prior art. An example of such a screen would be to use a library in accordance with the present invention against a receptor. In this way one might be able to select for a peptide that mimics the native ligand. This peptide could then be produced using standard Merrifield synthesis, and the synthetic peptide may be used as either a lead peptide for further development or directly as a modulator of the receptor being studied. For example, a random phage display peptide library may be used to screen against HIV gp120. In this way, one might be able to select for a peptide that binds to gp120 at precisely the CD4 binding site. Such a peptide would be expected to bind to the virus and to prevent the association of the virus to the CD4 receptor and, thus, prevent infection. One could also produce a library derived from CD4 itself and hope to discover a fragment of CD4 that binds to gp120 and acts as a decoy. All possibilities stem from the fact that the library is produced in the genetically stable expression system of the present invention.

As to the selectable marker, a particular advantage of placing this marker between the two pVIII genes is to create a situation that if, for whatever reason, there is a genetic recombination based on the limited homology of the native pVIII gene and the recombinant pVIII gene, then the selectable marker would be excised and lost as well. Running an experiment in the presence of the antibiotic would result in loss of those recombinants that have lost their resistance gene. Other markers that could be used in place of the tetracycline resistance gene would be the ampicillin resistance gene, β-lactamase or chloramphenicol resistance gene.

As stated hereinabove, it is also possible within the scope of the present invention to also insert an exogenous gene between the pIII gene and its promoter. By doing so, the phage can display a library of random peptides on either pIII or pVIII and a marker on the other, such as green fluorescence protein. This would make the selection of the bound phage easier or amenable to high throughput screening. The new exogenous gene can either be inserted directly between the pIII gene and its promoter or a second pIII gene can be inserted similarly to that discussed in the present invention for the second pVIII gene in order to ensure that the native pIII gene will always be produced along with the second pIII gene and the peptide of interest. It is not necessary to insert a separate pIII gene as, in contrast to pVIII, pIII can tolerate large inserts.

The following examples are directed to preferred embodiments in accordance with the present invention and show how to make and use the present invention. Those of ordinary skill in the art will understand, however, that these examples do not detract from the breadth of the present invention as described herein and that appropriate substitutions can be made without engaging in undue experimentation by those of ordinary skill in the art.

Materials and Methods

Bacterial Strains, Phages, Reagents, and General Techniques

E. coli strains used in this study were the following. K802: F⁻ e14⁻(McrA⁻) lacY1 or Δ(lac)6 supE44 galK2 galT22 rfbD1 metB1 mcrB1 hsdS3(r_{K}⁻ m_{K}⁻). K91KAN: a derivative of K91 (Hfr-Cavalli, thi) in which the “mini-Kan hopper” element was inserted in the lacZ gene of K91 rendering this strain kanamycin resistant (Parmley et al, 1988). DH5α: F⁻ endA1 hsdR17(r_{K}⁻ m_{K}⁺) supE44 thi-1 recA1 gyrA(Nal^{r}) relA1 Δ(lacIZYA-argF)U169 deoR (Φ80dlacΔ(lacZ)M15). DH5αF′: F′ endA1 hsdR17(r_{K}⁻ m_{K}⁺) supE44 thi-1 recA1 gyrA(Nal^{r}) relA1 Δ(lacIZYA-argF)U169 deoR (Φ80dlacΔ(1acZ)M15). JM109: F′[traD36 lacI^{q} Δ(lacZ)M15 proA⁺B⁺] e14⁻(McrA⁻) Δ(lac-proAB) thi gyrA96(Nal^{r}) endA1 hsdR17(r_{K}⁻ m_{K}⁺) relA1 supE44 recA1. XL1-Blue: F′[::Tn10 proA⁺B⁺ lacI^{q} Δ(lacZ)M15] recA1 endA1 gyrA96(Nal^{r}) thi hsdR17(r_{K}⁻ m_{K}⁺) supE44 relA1 lac. MC1061: F⁻ araD139 Δ(ara-leu)7696 galE15 galK16 Δ(lac)X74 rpsL(Str^{r}) hsdR2(r_{K}⁻ m_{K}⁺) mcrA mcrB1. NM554: F⁻ araD139 Δ(ara-leu)7696 galE15 galK16 Δ(lac)X74 rpsL(Str^{r}) hsdR2(r_{K}⁻ m_{K}⁺) mcrA mcrB1 recA13.

The wild type fd filamentous phage and fd-tet vector were kindly provided by G. P. Smith (University of Missouri, Columbia, Mo.). The M13K07 phage was purchased from New England BioLabs Inc., MA (NEB). DNA was isolated using the alkaline lysis procedure and purified on a cesium chloride gradient as described previously (Stern et al, 1998). All the restriction enzymes used were purchased from NEB and the digestions were performed following the manufacturer's instructions. The monoclonal antibody GV4H3 was produced from a Balb/c mouse immunized with the HIV-1 envelope protein gp120 (Denisova et al, 1996).

Oligonucleotides

The oligonucleotides used in this study were the following.

| | | |
|---|---|---|
| ON1: | 5′-GCCTTCGTAGTGGCATTACG-3′ | (SEQ ID NO: 6) |
| ON2: | 5′-AGCCCGCTCATTAGGCGGGCTTCATTACCGGCCACGTCGGCCAC | (SEQ ID NO: 7) |
| | CCTCAGCAGCGAAAGAC-3′ | |
| ON3: | 5′-CGGTAATGAAGCCCGCCTAATGAGCGGGCTTTTTTTTGGCCTCT | (SEQ ID NO: 8) |
| | GGGGCCGATCCCGCAAAAGCGGCC-3′ | |
| ON4: | 5′-GGTCAGACGATTGGCCTTG-3′ | (SEQ ID NO: 9) |
| ON5: | 5′-GAGCTCGCTAGCGCTCGAGCCAAAAAAAAAGGCTCCAAAAGG-3′ | (SEQ ID NO: 10) |
| ON6: | 5′-CTAGAGCAGGGTCCAGCTAA-3′ | (SEQ ID NO: 11) |
| ON7: | 5′-CTGGACCCTGCT-3′ | (SEQ ID NO: 12) |
| ON8: | 5′-TCGAGCTGTTGACAATTAATCATCGGCTCGTATAATGTGTGGAA | (SEQ ID NO: 13) |
| | TTGTGAGCGGATAACAATTGAGCT-3′ | |
| ON9: | 5′-CAATTGTTATCCGCTCACAATTCCACACATTATACGAGCCGATG | (SEQ ID NO: 14) |
| | ATTAATTGTCAACAGC-3′ | |
| ON10: | 5′-CCGTGCATCTGTCCTCGTTC-3′ | (SEQ ID NO: 15) |
| ON11: | 5′-CAGGCTTAAGCATCGACGTCTTATCAAGACGCCTTGCTTGTAA | (SEQ ID NO: 16) |
| | ACTTTTTGAATAACTTGATACCGATAGTTGCGC-3′ | |
| ON12: | 5′-GTCTTGATAAGACGTCGATGCTTAAGCCTGGCTAGCCATCAGAT | (SEQ ID NO: 17) |
| | CTGAGTCGGCCGCTGTTTAAGAAATTCACCTCG-3′ | |
| ON13: | 5′-GCCGTACCGCTAGCATTAGAAAAACTCATCGAGC-3′ | (SEQ ID NO: 18) |
| ON14: | 5′-GCTTCCTGACAGGAGGCCGTTTTGTTTTGCAGCCCACCTGAGCT | (SEQ ID NO: 19) |
| | CCCAGCTTAAGGTGTCTCAAAATCTCTGATG-3′ | |
| ON15: | 5′-GGCTGAGGGACGTCGAGGGCATGCGTACCCGATAAAAGCGGCTT | (SEQ ID NO: 20) |
| | CCTGACAGGAGGCCG-3′ | |
| ON16: | 5′-AGCCTTTCAGGTCAGAAGGG-3′ | (SEQ ID NO: 21) |
| ON17: | 5′-ATTCATGCCGGAGAGGGTAG-3′ | (SEQ ID NO: 22) |
| ON18: | 5′-CATAACACCCCTTGTATTACTG-3′ | (SEQ ID NO: 23) |
| ON19: | 5′-GCTTACATAAACAGTAATACAAGGGGTGTTATGAATAGTTCGAC | (SEQ ID NO: 24) |
| | AAAGATCGC-3′ | |
| ON20: | 5′-GGATCGAGAGCTAGCATAACTAAGCACTTGTCTCCTG-3′ | (SEQ ID NO: 25) |
| ON21: | 5′-TGCCGGCGCTCCCGCTGGTTTCGCTATCCTGTCAGCACCCGGGT | (SEQ ID NO: 26) |
| | CTG-3′ | |
| ON22: | 5′-ACCCGGGTGCTGACAGGATAGCGAAACCAGCGGGAGCGCCGGCA | (SEQ ID NO: 27) |
| | CGT-3′ | |

Vector Construction

In this study two novel vector-systems were constructed. The rationale for their compositions and structures is described in the Results. Detailed diagrams of the vectors and sub-structures are given in the Figures as indicated. The following are the details of the specific steps taken to construct the vectors.

Construction of ftac88

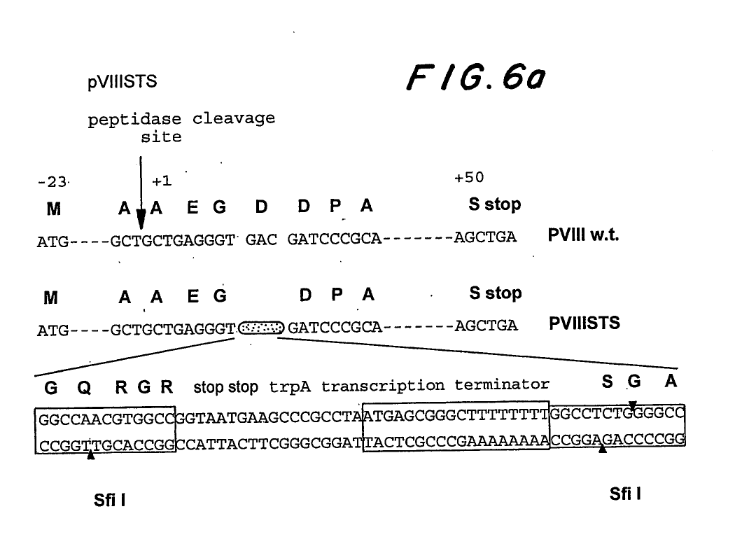

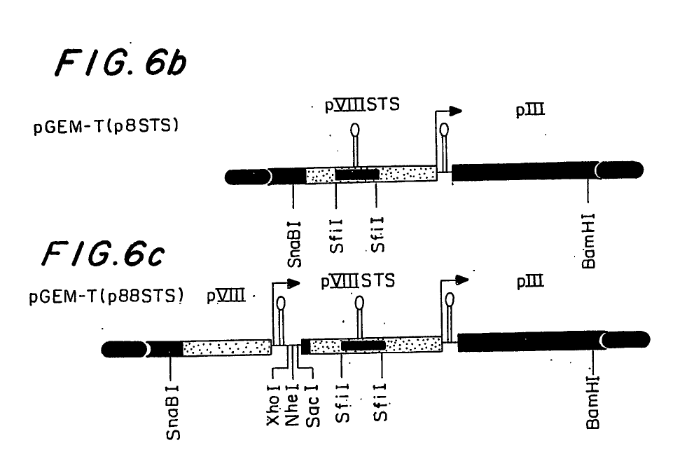

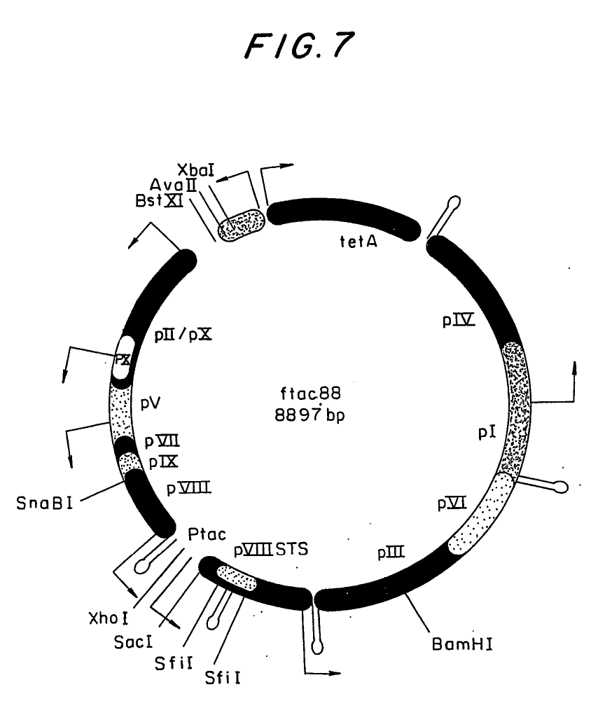

The ftac88 vector (for detailed map see FIG. 7) was constructed through the following steps. First, the recombinant pVIII gene, designated pVIIISTS (for orientation see FIG. 6A), was generated by introducing a 62 bp insert three codons downstream to the leader peptide using the “SOEing” PCR mutagenesis (Horton et al, 1990). For this, four oligonucleotides were used. ON1 and ON2 were used to PCR amplify from fd a 169 bp fragment. ON3 and ON4 were used to PCR generate from fd a 899 bp fragment. ON2 and ON3 each contain 5′-extension corresponding to the 62 bp insert. Thus, the resulting PCR fragments contain an identical 30 bp stretch of the novel sequence. The two fragments were purified from agarose gel, mixed and used as a combined template for PCR amplification using the oligonucleotides ON1 and ON4 to generate the final 1071 bp product containing the modified pVIII gene, part of pIII gene, and the flanking BamH I-SnaB I sites. The PCR reaction was performed by using the Taq polymerase which adds adenine to the 3′ ends of PCR products and thus the resulting 1071 bp fragment was directly ligated with the linearized pGEM-T vector (Promega Corporation, WI) containing complementary 3′ thymidine overhangs, generating the pGEM-T(p8STS) construct (see FIG. 6B).

Next, the wild type pVIII gene was PCR amplified from fd using the oligonucleotides ON1 and ON5. ON5 has a 5′ extension that contains the restriction sites Xho I, Nhe I and Sac I. The PCR reaction was performed using the Pwo Polymerase (Boehringer Mannheim), resulting in blunt-ended 335 bp fragment from the SnaB I site to just beyond the overlapping promoter/terminator followed by the ON5 introduced restriction sites. The resulting fragment was ligated with the blunt-ended SnaB I linearized pGEM-T(p8STS) construct generating the pGEM-T(p88STS) construct (see FIG. 6C).

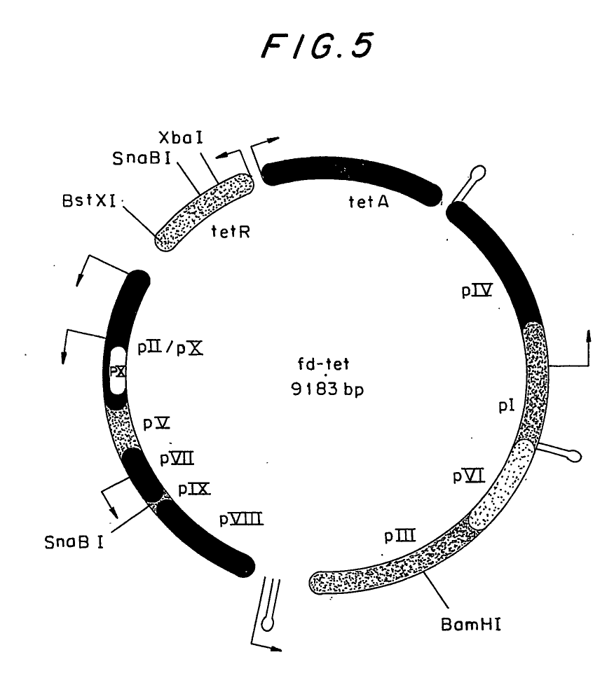

To remove the SnaB I site in tetR gene, the fd-tet vector (for detailed map see FIG. 5) was digested with Xba I and BstX I restriction enzymes and the 8458 bp fragment was purified from agarose gel. The purified fragment was ligated with a linker containing the Ava II site produced by annealing the two complementary oligonucleotides ON6 and ON7.

To exchange the SnaB I-BamH 1952 bp segment of fd-tet for the SnaB I-BamH 11378 bp segment of pGEM-T(p88STS) construct containing both the wild type and recombinant pVIII genes, the Ava II containing fd-tet derivative was digested with SnaB I/BamH I and the 7519 bp fragment was purified from agarose gel. In the same manner, the pGEM-T(p88STS) construct was digested with SnaB I/BamH I and the released 1378 bp fragment was purified from agarose gel. These two fragments were ligated to generate an intermediate vector.

This intermediate vector was digested with Sac I and Xho I restriction enzymes and the 13 bp stuffer was removed by applying the DNA digest on a chroma-spin™ column (Clontech Laboratories, Inc., Palo Alto, Calif.). The linearized intermediate vector was ligated with a linker containing the tac promoter produced by annealing the two complementary oligonucleotides ON8 and ON9, generating the ftac88 vector.

Construction of fth1

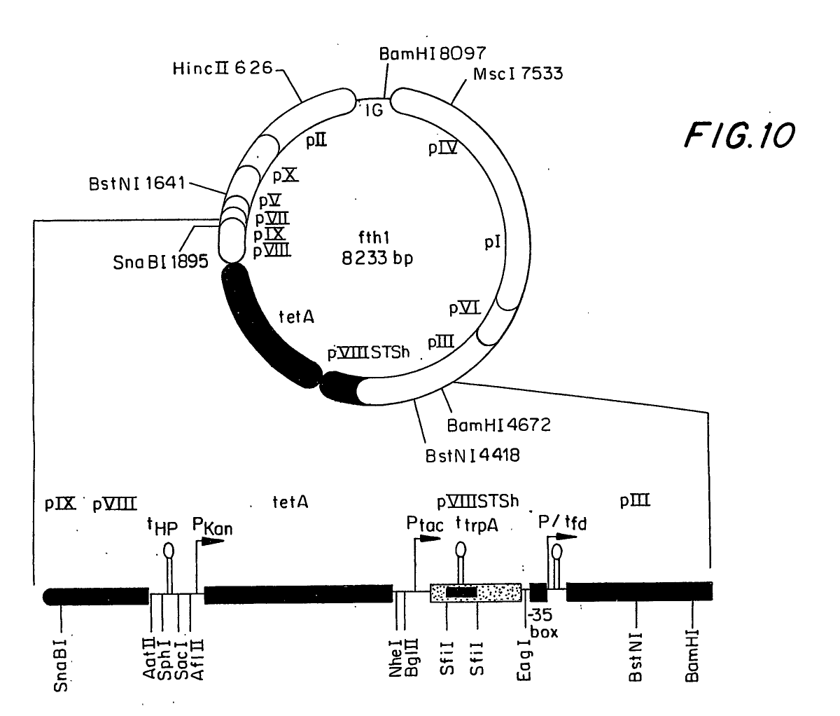

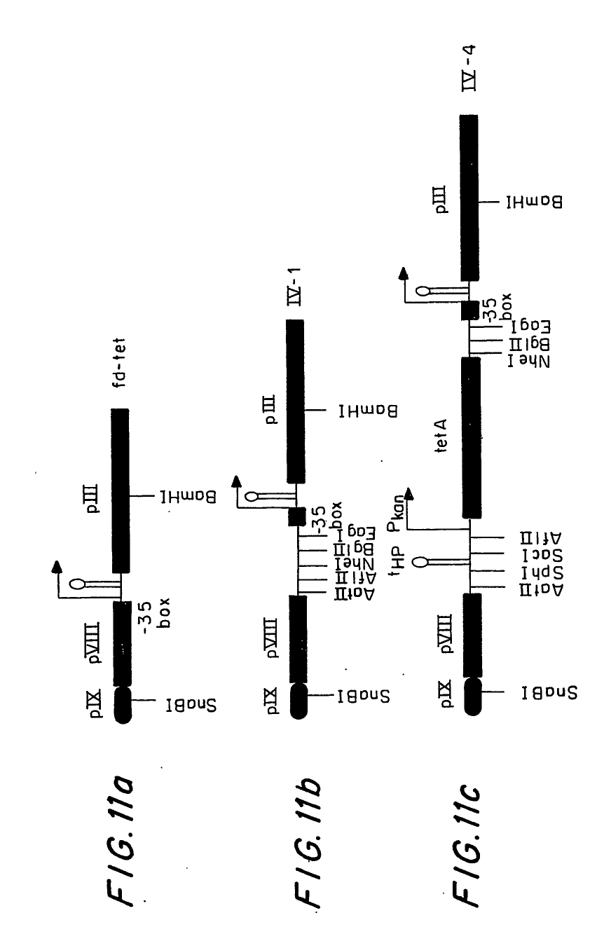

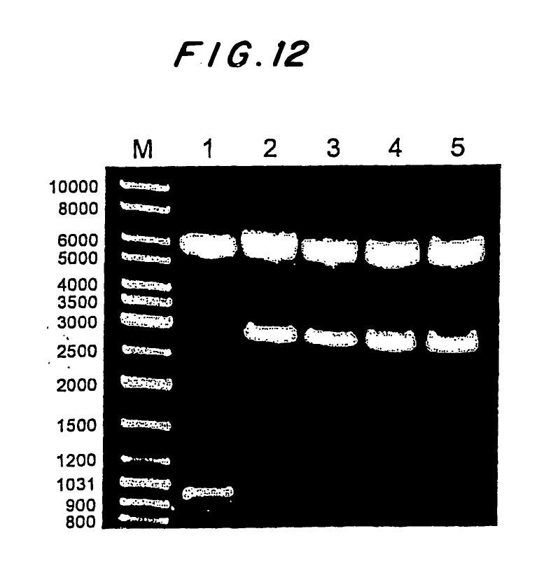

The construction of the fth1 vector (for detailed map see FIG. 10) was performed through a series of intermediate vectors. The intermediate vector-1 (IV-1, see FIG. 11B) was constructed by introducing a DNA segment ten codons upstream to the stop codon of the wild type pVIII gene using the “SOEing” PCR mutagenesis described above. Two fragments of 519 bp and 827 bp were PCR generated from fd using the oligonucleotides ON10/ON11 and ON12/ON4 respectively. The two fragments were mixed and used as a combined template using ON10/ON4 to PCR generate the final 1316 bp fragment. This product was digested with SnaB I and BamH I and the resulting 1034 bp fragment was purified from agarose gel and used to replace the corresponding SnaB I-BamH I fragment of the Ava II containing fd-tet derivative described above for the construction of ftac88.

Next, the IV-2 was constructed by introducing into the IV-1, downstream to the wild type pVIII gene, the novel HP transcription terminator followed by the kanamycin resistance gene. This was performed in order (i) to functionally separate the two phage-transcriptional units and (ii) to enable the reconstitution of the intergenic region while maintaining an alternative antibiotic selectable marker (i.e. kanamycin). The kanamycin resistance gene was PCR amplified from M13K07 using the oligonucleotides ON13 and ON14. Both oligonucleotides contain 5′ extensions enabling the incorporation of additional flanking sequences. The 3′ addition contained the Nhe I restriction site and the 5′ extension contained additional cloning sites adjacent to the kanamycin promoter and part of the HP terminator. The resulting 944 bp fragment was purified from agarose gel and used as a template for PCR amplification using the oligonucleotides ON13 and ON15. The latter has a 5′ extension containing the second part of the HP terminator and additional cloning sites ending with the Aat II site. Thus the resulting 984 bp fragment contains the entire HP terminator bracketed between multiple cloning sites followed by the kanamycin resistance gene. This product was digested with Nhe I and Aat II and the resulting fragment was purified from agarose gel. The purified fragment was ligated with Nhe I/Aat II digested IV-1 vector after removing the 20 bp stuffer by applying the vector digest on a chroma-spin™ column, ultimately generating IV-2.

The IV-2 was used to reconstitute the intergenic region containing the origins of replication. This was performed by PCR amplifying the intergenic region from fd phage using the oligonucleotides ON16 and ON17. The resulting 1081 bp fragment containing the Msc I-Drd I segment was digested with Msc I and Drd I restriction enzymes. The 679 bp Msc I-Drd I product was purified from agarose gel and used to exchange the Msc I-Drd I fragment in IV-2 containing the tet fragment, generating IV-3.

The open reading frame only of the kanamycin resistance gene was replaced by the open reading frame of the tetracycline resistance gene thus constructing the IV-4 vector (see FIG. 11C). This was performed using the “SOEing” PCR. The oligonucleotides ON1 and ON18 were used to PCR amplify from IV-2 the 463 bp fragment containing the upstream sequence to the ATG initiating codon of the kanamycin gene. The oligonucleotides ON19 and ON20 were used to PCR amplify from fd-tet the 1265 bp fragment containing the open reading frame of the tetracycline resistance gene. The two fragments were mixed and used for PCR amplification of the 1698 bp fragment using the oligonucleotides ON1 and ON20. The PCR was designed to flank the resulting 1698 bp fragment between Nhe I and Sac I restriction sites enabling the digestion of this fragment with the corresponding enzymes. After purifying the digested fragment from agarose gel, it was used to replace the corresponding Nhe I-Sac I fragment of IV-3.

The final step was to generate the synthetic recombinant pVIII gene using a panel of overlapping oligonucleotides (not shown). Part of the panel was used as templates and the other part as 5′-extended primers for PCR amplification thus generating separately small pieces of DNA. These small fragments were sequentially mixed and used as a combined template for the “SOEing” PCR ultimately generating the recombinant pVIIISTSh gene that subsequently was introduced between the Bgl II/Eag I sites of IV-4 producing the fth1 vector.

Introduction of the GV4H3 Epitope into ftac88 and fth1 Vectors

The vectors ftac88 and fth1 were digested with Sfi I restriction enzyme and the 49 bp stuffer containing the trpA terminator (for orientation see FIG. 6a) was removed by applying the DNA digest on a chroma-spin™ column. The purified linear vector was ligated with GV4H3 epitope encoding linker produced by annealing the two complementary oligonucleotides ON21 and ON22.

Dot Blot Analysis of GV4H3 Epitope Presenting Phages

The phages were applied via a vacuum manifold to a nitrocellulose membrane filter. After blocking (5% evaporated spray dried skim milk 1.5% fat in Tris buffered saline (TBS)) for 1 hour, the membrane was washed briefly with TBS and incubated overnight with 1 μg/ml of GV4H3 mAb in TBS/5% milk at 4° C. with gentle rocking. After washing, the membrane was incubated with goat-anti mouse IgG/HRP conjugate diluted 1:5000 in TBS/5% milk for 1 hour at room temperature. The positive signals were detected by ECL (Amersham International plc, Buckinghamshire, England) immunodetection.

### Results

The fd-tet vector of Smith (Zacher et al, 1980) was used as a starting point for our studies.

The fd-tet Expression Vector

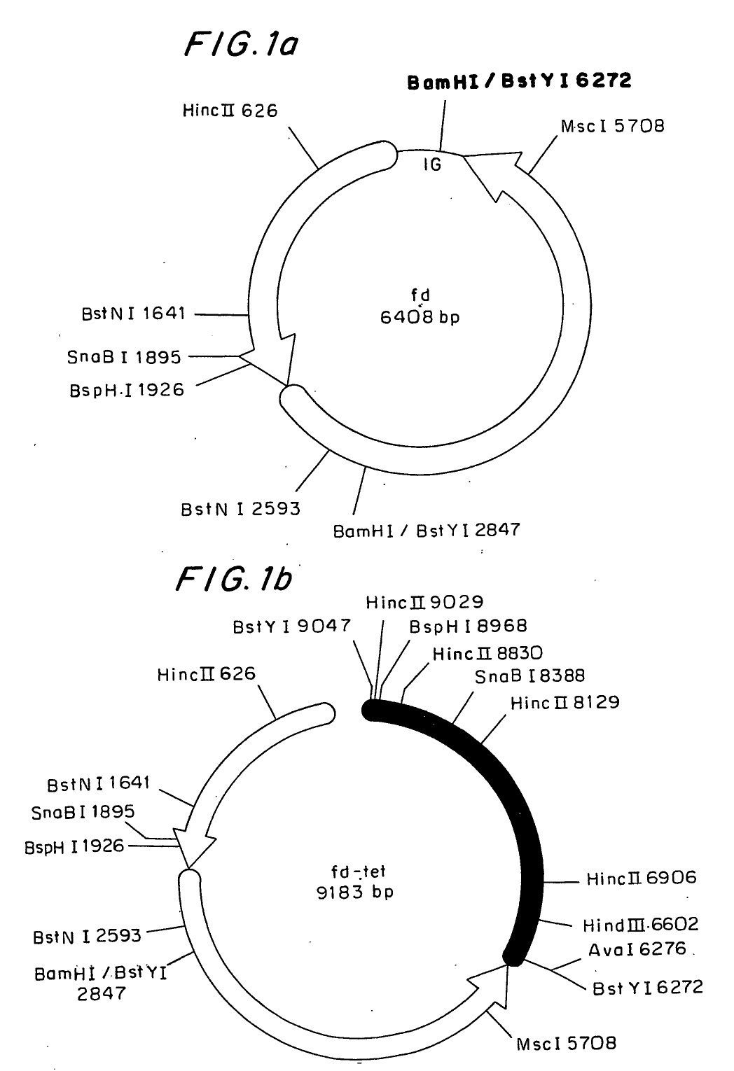

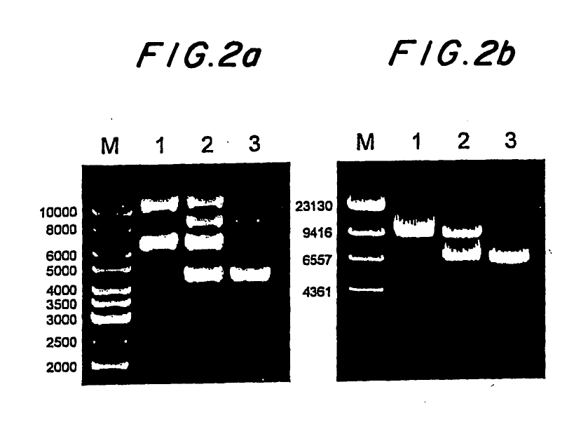

Using the fd genome as a scaffold molecule, Zacher et al (1980) constructed the fd-tet phage, which contains the tetracycline resistance gene as a selectable marker (see FIG. 1). The fragment coding for the tetracycline resistance was obtained from the transposon Tn10 (a 2775 bp fragment flanked between two Bgl II sites). This segment was introduced into the BamH I site of the intergenic region containing the origins of replication of the fd genome. The fd-tet vector has the following advantages:
1. fd-tet phages confer selectable tetracycline resistance to infected or transfected host bacteria.
2. The tet fragment introduces unique restriction sites that can be exploited for cloning foreign DNA sequences.

Thus, fd-tet was used, for example, as a cloning vector by exploiting the unique Hind III site, situated in the tet fragment, to clone Hind III digested phage X DNA (Zacher et al, 1980). Furthermore, Smith has reported the introduction of a second pVIII gene in the same region generating a “type 88” vector designated f88.4, and the successful production of pVIII mosaic phages (Zhong et al, 1994).

However, a point for concern is the report that foreign DNA, cloned into the intergenic region of filamentous phages, tends to be unstable (Model et al, 1988; Sambrook et al, 1989; Vieira et al, 1987). Occasionally, parts of the cloned DNA are deleted and the rate of the deletion event tends to be directly correlated with the size of the inserted fragment. Therefore, the question of the genetic stability of DNA inserts cloned into the intergenic region was examined.

Genetic Instability in fd-tet Vector

Purified fd-tet dsDNA generated two distinct electrophoretic patterns on agarose gels when isolated from two different E. coli strains (K802 and K91KAN). The DNA isolated from K802 exhibited the supercoiled and relaxed forms of the fd-tet genome (FIG. 2A, lane 1) whereas the DNA isolated from K91KAN contained the supercoiled and relaxed forms of two distinct DNAs (FIG. 2A, lane 2). One DNA corresponded to the fd-tet genome while the second compared well with the parent fd genome (FIG. 2A, lane 3). The two purified fd-tet preparations and fd were then digested with the Msc I restriction enzyme that should generate full length linear DNA for both fd and fd-tet (the Msc I restriction site is unique in both fd and fd-tet, see FIG. 1). Digestion of fd-tet from K802 and fd each generated only one band with the expected linear sizes of 9183 bp and 6414 bp respectively (FIG. 2B, lanes 1 and 3). However, digestion of fd-tet derived from K91KAN generated two bands, one indistinguishable in mobility from the linear fd-tet and the second corresponding in size to linear fd (FIG. 2B, lane 2). This indicates that for a substantial amount of DNA, a deletion of approximately 2.8 kb occurred when the fd-tet genome was produced in K91KAN bacteria.

Two points, therefore, must be addressed:
1. What DNA sequences are lost through the deletion process?
2. How are the differences in the bacterial strains related to the apparent genetic instability?
   Deletion of the tet Fragment

As the deleted DNA was 2.8 kbp, it was postulated that it might correspond to the tet-fragment (2775 bp) residing in the intergenic space of fd-tet (see FIG. 1). Therefore, the SnaB I/Ava I digests of fd-tet DNA derived from both bacterial strains as before were compared. The SnaB I site is unique in fd, however, it appears twice in fd-tet while the Ava I site exists only in the tet fragment of fd-tet. The SnaB I/Ava I double digestion of fd-tet isolated from K802 generated three fragments with the expected sizes: 4381 bp, 2690 bp and 2112 bp (FIG. 3, lane 1). The double digestion of the K91KAN derived fd-tet DNA, however, generated four DNA fragments (FIG. 3, lane 2); the three fragments corresponding to fd-tet and an additional fragment corresponding to the deletion product similar in size to a single cut full length 6414 bp fd (FIG. 3, lane 3, compare also with FIG. 2B). This indicates that the SnaB I-Ava I fragment of the tet insert in the intergenic region was deleted from fd-tet.

This conclusion was further supported by BspH I/Hind III digestion of fd-tet derived from both bacteria. The Hind III site is unique to fd-tet (see FIG. 1). The BspH I site appears only once in fd and twice in fd-tet (see FIG. 1, the second BspH I site is situated in the tet fragment). The BspH I/Hind III digestion of fd-tet DNA isolated from K802 generated the expected three fragments of 4676 bp, 2366 bp, and 2141 bp (FIG. 3, lane 4). However, the BspH I/Hind III double digestion of fd-tet DNA isolated from K91KAN generated not only the three fd-tet derived fragments but also a fourth fragment (approximately 6.4 kb) corresponding in size to single cut full-length fd (FIG. 3, lane 5, note arrow) derived from the deletion product. This not only confirms the selective deletion of the tet fragment but extends the boundaries of the deletion beyond the SnaB I site to at least the BspH I site (see FIG. 1).

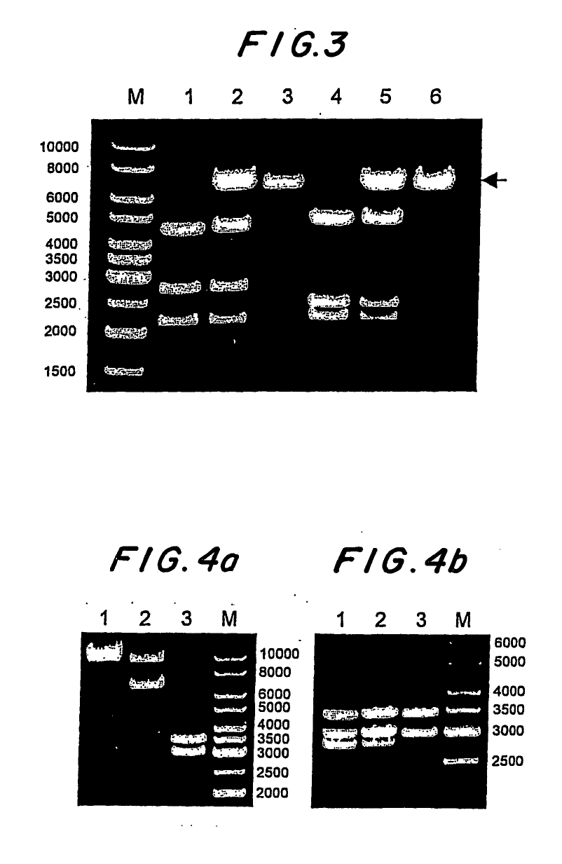

Is the deletion a precise reversion to fd? If so, one would reconstitute the original BamH I site used to clone the tet fragment, and thus generate two restriction fragments (3425 bp and 2989 bp as in the fd BamH I digest, see FIG. 4A lane 3) at the expense of the linearized deletion product (see for example the Msc I digest in FIG. 2B lane 2). As can be seen in FIG. 4A (lane 2), BamH I digestion of fd-tet isolated from K91KAN produced a pattern similar to that shown for Msc I digest. Two bands corresponding to the linearized fd-tet (9183 bp) and the full-length linearized deletion product (approximately 6.4 kb) were generated. This thus illustrates that although the deletion encompasses much or all of the tet fragment, it does not precisely regenerate the second BamH I site of the fd parent molecule.

Thus, the next question addressed, once the 2.8 kbp deletion was mapped to the vicinity of the tet fragment, was whether or not the excision sites lay within or outside of the tet fragment. For this the BstY I digest illustrated in FIG. 4B was performed (two BstY I sites, created during the fd-tet construction, precisely bracket the tet fragment, see FIG. 1). The gel pattern obtained for the BstY I digest of fd-tet derived from K802 had three fragments (3425 bp, 2983 bp and 2775 bp) identical in size to those generated when K91KAN derived DNA was used (FIG. 4B, compare lane 1 with lane 2). This demonstrates that at least one of the two BstY I sites was retained in the deleted product. Therefore, sequences immediately adjacent to the original BamH I site, must also be preserved in the deleted fd-tet, a fact that may be relevant to the mechanism of the deletion process (see Discussion).

The Deletion is recA Independent and F+Dependent

K802 and K91KAN strains of E. coli were introduced by Smith to produce and use the phage display vectors he has pioneered (Parmley, 1988). The principle consideration is that phage infectability is dependent on the F-factor. Therefore, manipulation of the DNA vector, its modifications and construction can be made in the absence of infection in F-bacteria (such as K802), thus avoiding multiply infected bacteria. Once the vector is completed, then in order to screen a phage library or to amplify and generate high titers of phages one must use F⁺ bacteria (e.g., K91KAN).

Table 1 illustrates that in a variety of E. coli strains tested, the tet deletions were detected only in the F⁺ strains. Most noteworthy is the comparison between the isogenic DH5α and DH5α F′ E. coli strains. Here too the deletion occurs exclusively in the DH5αF′ strain. These strains are identical in their genotypes except that DH5αF′ carries the F-factor. This indicates that the deletion is F-factor dependent. Moreover, both strains are RecA⁻ in which the recA gene is mutated and thus classical homologous recombination is not possible. Therefore, the deletion event is recA independent.

TABLE 1
The Deletion is F⁺ Dependent and recA Independent

| E. coli Strain | redA | F-episome | Deletion |
|---|---|---|---|
| K802 | + | − | − |
| K91KAN | + | + | + |
| DH5α^{a} | − | − | − |
| DH5αF′^{a} | − | + | + |
| JM109 | − | + | + |
| XL1-Blue | − | + | + |
| MC1061^{b} | + | − | − |
| NM554^{b} | − | − | − |

^{a}DH5α and DH5αF′ are isogenic strains; DH5αF′ contains the F-episome
^{b}MC1061 and NM554 are isogenic strains; NM554 is recA⁻

As the F-factor is essential when using filamentous phages, working with F⁺ bacterial strains cannot be avoided. Therefore, introducing the recombinant pVIII gene in a region prone to F⁺ dependent deletions is potentially problematic. The suitability of the non-coding region between the wild type pVIII gene and the pIII gene as a site for genetic manipulation and introduction of a second pVIII gene was therefore explored.

Construction of the ftac88 Vector

Designing a phage display vector with the aim of introducing a second pVIII gene in the non-coding region between the wild type pVIII and pIII genes presents two obstacles that must be considered.
1. Unique restriction sites for cloning are unavailable in this region.
2. This region contains an essential overlapping promoter and rho-independent transcription terminator that are inseparable (see FIG. 5;
see also Van Wezenbeek et al, 1980).

The terminator terminates the transcription of upstream genes ending with the pVIII gene, whereas the promoter initiates the transcription of downstream genes starting with the pIII gene. Therefore, the strategy adopted to insert the recombinant second pVIII gene was to duplicate this promoter/terminator as well. Ultimately, a “type 88” expression vector (designated ftac88) was constructed as follows:

The first step was to generate the modified pVIII gene. This was designed to contain unique restriction sites that could be used to introduce foreign DNA sequences and thus enable the presentation of their corresponding encoded peptides at the N-terminus of the pVIII protein. Such a modified pVIII gene, designated pVIIISTS, was produced by “SOEing” PCR (Horton et al, 1990) mutagenesis in which the GAC codon for residue Asp, at position 4 of mature wild type pVIII protein, was replaced by an insert of 62 base pairs (“STS” insert, FIG. 6A). This insert contained two Sfi I sites that bracketed two translation stop-codons followed by the trpA transcription terminator. Thus, the transcription of the pVIIISTS gene terminates prematurely, rendering the expression of this gene silent. In order to express this pVIII gene one must exchange the Sfi I flanked stuffer for a functional in-frame insert of DNA. The PCR was designed to generate a fragment corresponding to the SnaB I-BamH I segment of fd-tet (see FIG. 5) that was cloned into the pGEM-T vector, designated pGEM-T(p8STS) (FIG. 6B).

Next, a wild type pVIII gene was cloned into the SnaB I site of pGEM-T(p8STS). This was achieved by PCR of the wild type pVIII gene segment from fd-tet starting from the upstream SnaB I site and continuing just beyond the overlapping promoter/terminator. The downstream antisense primer incorporated three unique restriction sites as well. The PCR product was cloned into the blunt ends of SnaB I cut pGEM-T(p8STS) thus generating the desired pGEM-T(p88STS). As is illustrated in FIG. 6C the SnaB I-BamH I fragment of pGEM-T(p88STS) contains two pVIII genes, duplicated promoter/terminator elements and several unique cloning sites.

To complete the generation of the “type 88” phage display vector all that was required was to replace the SnaB I-BamH I segment of fd-tet with the corresponding SnaB I-BamH I fragment of pGEM-T(p88STS). However, fd-tet contains two SnaB I sites (see FIG. 5). Therefore, the SnaB I site in the tetR gene of fd-tet was removed. For this, fd-tet genome was digested with Xba I/BstX I to remove the 725 bp fragment containing the SnaB I site that was replaced by a linker containing an Ava II site. This intermediate modified fd-tet was then digested with SnaB I/BamH I and the wild type segment was exchanged for the correspondingly cut fragment derived from pGEM-T(p88STS). Furthermore, a tac promoter was introduced between the Xho I and Sac I sites to produce the “type 88” vector designated ftac88 shown in FIG. 7.

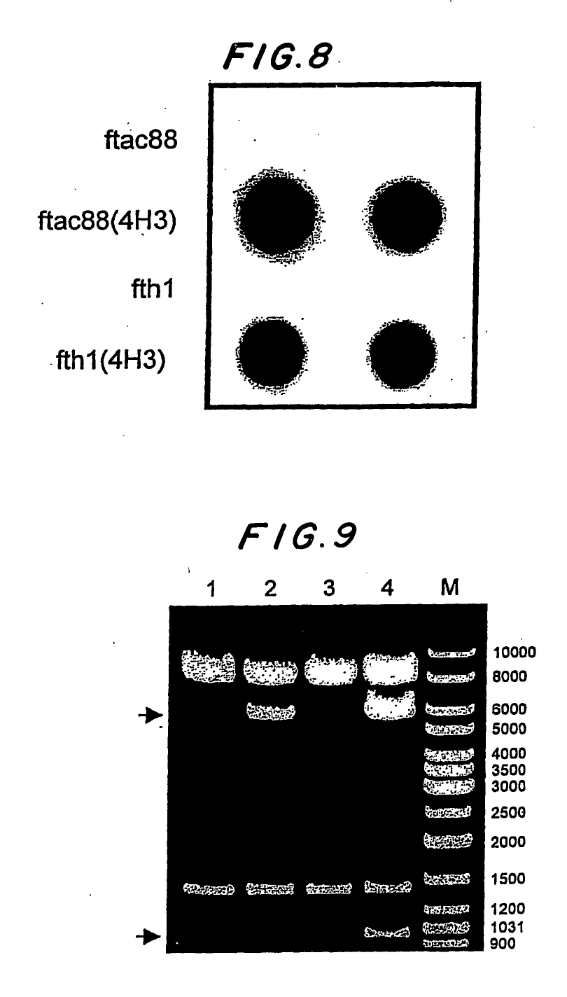

It was then necessary to evaluate whether or not the ftac88 vector can produce mosaic phages displaying both wild type and chimeric pVIII proteins. For this a linker encoding the linear peptide APAGFAIL (SEQ ID NO:28) (the epitope corresponding to the anti-gp120 mAb GV4H3 (Denisova et al, 1996)) was inserted in frame between the two Sfi I sites, eliminating the trpA terminator. As demonstrated by the dot blot analysis (FIG. 8), this construct (ftac88(4H3)) produced phages that were recognized by the GV4H3 mAb.

Genetic Instability in ftac88

The orientation of the wild type pVIII and the modified pVIIISTS genes in the ftac88 vector is a direct tandem repeat. The construction of this vector was performed in recA⁻ bacteria and as such excludes the possibility of recA dependent homologous recombination. However, in view of the fact that the deletion of the tet fragment in fd-tet (described above) is recA independent and F⁺ dependent, the genetic stability of the modified pVIIISTS gene in DH5αF′ was examined. For this, ftac88 DNA preparations from DH5α and DH5αF′ bacteria were compared.

Double digestion of ftac88 with SnaB I and BamH I should release a 1378 bp fragment containing both pVIII genes (see FIG. 7). Indeed, SnaB I/BamH I double digestion produced only the expected 1378 bp fragment, regardless of the source of the DNA (FIG. 9, lane 1, 2). As expected, the deletion of the tet fragment still occurred in DH5αF′ (FIG. 9, lane 2; indicated by the upper arrow). Thus one can conclude that the modified pVIIISTS gene is genetically stable in ftac88.

However, the purpose of the ftac88 vector is to display peptides as chimeric pVIII proteins and as such, it was important to further investigate the genetic stability of the modified pVIIISTS gene when it contained a peptide coding sequence rather than the stuffer with the trpA terminator. For this, the ftac88 DNA construct expressing the GV4H3 epitope (above) was prepared from DH5α and DH5αF′ bacteria and used for the double digestion, SnaB I/BamH I analysis, as above. Here the situation was surprising. Digestion of the DNA derived from DH5α released as expected a 1376 bp fragment (FIG. 9, lane 3). However, SnaB I/BamH I digestion of the same DNA prepared from DH5αF′ generated an additional smaller fragment of approximately 950 bp (FIG. 9, lane 4). This indicates that a deletion of approximately 400 bp occurred. This deletion corresponded to selective loss of the recombinant pVIII gene and reconstitution of the wild type pVIII-pIII configuration as confirmed by sequence analysis (not shown). Therefore, it was concluded that the modified pVIIISTS gene, when containing coding sequences, becomes genetically unstable in DH5αF′. This also suggests that the stability of the ftac88 observed in DH5αF′ is rendered due to the trpA terminator which could imply that the deletion process may require transcribed regions.

In summary, it is clear that the non-coding region between pVIII and pIII can be used. Moreover, the obstacle of the overlapping promoter/terminator can be overcome. However, genetic instability of the recombinant pVIII gene was still a problem.

Construction of fth1 Vector

In light of the above experience in designing, constructing and testing the ftac88 vector, it was decided to redesign a novel “type 88” vector that would overcome the problems of genetic instability and include other improvements. The attributes of such a vector, designated fth1 (FIG. 10) are listed herewith:
3. The second pVIII gene contained the 62 bp “STS” insert as in ftac88. However, the gene was constructed using synthetic oligonucleotides to generate a pVIII protein of identical amino acid sequence yet coded for with alternative codon usage. This obviated the formation of tandem repeats, a configuration that is prone to deletion events as illustrated in the analysis of ftac88 (above). The homologous gene was designated pVIIISTSh. The pVIIISTSh gene was cloned, as in ftac88, between the wild type pVIII and pIII genes
4. To avoid the generation of additional tandem repeats, an alternative HP terminator (Nohno et al, 1986) was introduced into the 3′ region of the wild type pVIII gene ten codons upstream to the pVIII stop codon. Moreover, the severed 3′ region coding for the C-terminal domain of the wild type pVIII protein was reconstituted using alternative codon usage (so to reduce homology within the −35 box of the promoter of the pIII gene residing in this region (Van Wezenbeek et al, 1980)).
5. The tetracycline resistance gene was cloned between the two pVIII genes. This was accomplished via PCR of the open reading frame of the tetracycline resistance gene only and placing it downstream to a kanamycin promoter (derived from M13K07 phage). This was done so as to avoid using the bi-directional tetracycline promoter (see FIG. 5) which would otherwise generate an anti-sense transcript of the wild type pVIII gene were it used in this new position. Also the exclusion of the tetracycline repressor is advantageous as the tetracycline resistance gene thus becomes constitutively expressed, making pre-incubation with low levels of tetracycline to induce resistance unnecessary.
6. The original intergenic space containing the ori sequences was reconstituted. Previously, the tet fragment used by Smith to construct fd-tet, was inserted into the fd ori(−) rendering this vector and its derivatives low copy number and thus producing relatively low phage titers (Smith, 1988). Reconstituting the intergenic region in fth1 provided two advantages: the copy number of the DNA was greatly increased and the titers of secreted phage increased as well (see below).

The construction of fth1 (see FIG. 11) was accomplished using “SOEing” PCR as described above enabling the introduction of multiple cloning sites and novel genes into the non-coding region between the wild type pVIII and pIII genes of fd-tet.

The first step was to modify fd-tet by the introduction of a short segment of DNA, ten codons upstream from the stop codon of the wild type pVIII gene (FIG. 11B). This segment not only provided novel restriction sites but also separated the promoter/terminator adjacent to the pIII gene from the wild type pVIII gene by reconstituting the severed last ten codons of the pVIII open reading frame (intermediate vector 1 (IV-1) in FIG. 11B).

Subsequently, additional intermediate vectors were produced (IV-2 and IV-3, see Materials and Methods) which eventually led to IV-4 (FIG. 1C). In the latter, the novel HP terminator was incorporated downstream to the reconstituted wild type pVIII gene followed by the tetracycline resistance open reading frame driven by the Kanamycin promoter. Moreover, the ori(−) in the intergenic region was also reconstituted in IV-4.

The final vector was produced by cloning the synthetic pVIIISTSh gene, driven by the tac promoter, into the Bgl II/Eag I digested IV-4 vector to give fth1 (see detailed map, FIG. 10).

The questions to be evaluated regarding the utility and advantages of fth1 therefore were:
1. Was the vector, and particularly the tetracycline resistance and recombinant pVIII genes, genetically stable?
2. Could the recombinant pVIII gene be used to stably express peptide inserts?
3. Was the fth1 a high copy number vector?
4. Could high titers of fth1 progeny be achieved?

In order to answer these questions, the GV4H3 epitope was introduced between the Sfi I sites in fth1 (as done previously for ftac88). This construct, called fth1(4H3), produced mosaic phages presenting the GV4H3 epitope that were recognized by the GV4H3 mAb (FIG. 8).

The stability of the tetracycline resistance gene and the modified pVIIISTSh gene was tested using restriction analyses as before. Digestion of fth1 and fth1(4H3) DNA, derived from DH5α or DH5αF′, with BstN I generated identical fragments as is illustrated in FIG. 12. BstN I sites appear twice in both fd (see FIG. 1) and fth1 (see FIG. 10). BstN I digestion of fd generated the expected two fragments of 5456 bp and 952 bp sizes (FIG. 12, lane 1). The latter contains the non-coding region between pVIII and pIII genes. As this region in fth1 contains the tetracycline resistance and the recombinant pVIIISTSh genes, the 952 bp fragment was shifted to generate the 2777 bp and 2775 bp bands in BstN I digests of fth1 and fth1(4H3) respectively derived from both DH5α and DH5αF′ (lane 2-5). Any deletions of either the tetracycline resistance gene or the recombinant pVIII gene or their parts would generate DNA fragments ranging in size from 952 bp to 2777 bp. No such fragments could be detected illustrating that the fth1 vector is genetically stable.

The functionality of the reconstituted ori(−) is directly apparent. In several independent purifications of fth1 DNA, 0.5-1 mg DNA/liter of bacteria was routinely obtained, as opposed to the typical yields of 50 μg DNA per liter of ftac88 harboring bacteria. Similarly a hundred fold increase is typically seen in phage titers for which 1012 phages/ml is measured for fth1 as compared to 10¹⁰ phages/ml achieved for fd-tet and its derivatives.

Thus the fth1 vector provides a convenient and stable “type 88” phage display expression system.

### Discussion

The selective deletion of the tet fragment from the intergenic space has been critically evaluated as a first step towards producing a novel “type 88” phage display vector. The present inventors have observed that in F⁺ bacterial strains a 2.8 kb fragment of DNA is excised from the region of the foreign tet insert, Smith originally introduced into the B-stem/loop of the ori(−) structure (Zacher et al, 1980). The loss of the insert's Ava I site, which overlaps the original remnant BamH I site, illustrates that downstream aspects of the insert are not preserved in the deleted form. The loss of the BspH I site and all four of the insert's Hinc II sites (not shown) argues that no more than 18 base pairs of the insert can be retained at the other extreme. Preservation of at least one BstY I site, yet lack of reconstitution of the original BamH I site, further stresses the close but not precise correspondence of the deleted fragment with the original tet insert.

As the insert is flanked by the inverted repeats that create the B-stem/loop and that these repeats are retained in the deleted form of the phage, one might postulate that the mechanism generating the deletion involves homology and base paring. Clearly, however, this can not be homologous recombination as that would require direct repeats of homologous stretches of over 50 bases and of course, a functional RecA protein in order to enable a deletion event (Clark, 1973; Matfield et al, 1985). The deletion events in our experiments occurred in recA⁻ bacterial strains. An alternative mechanism, the “slippage mechanism” proposed by Lovett (Feschenko et al, 1998), also requires direct repeats and therefore can not explain the deletion. To the best of our knowledge, no known mechanism can explain the deletion of the tet fragment as observed.

One might imagine that during the course of replication, the inverted repeats, flanking the tet fragment, align to form a B-stem/loop of sorts, greatly distorted by a grossly extended “bubble” (where the BamH I site had been), consisting of the 2.8 kb insert. During replication, the polymerase might be able to “skip” from one side of the bubble to the other thus omitting the entire insert and generating the deletion. Assuming that this is a very rare event one must still address the fact that in K91Kan cells over 50% of the DNA isolated was of the deleted form. Moreover, why is the deletion F⁺ dependent?

The matter of copy number might be explained due to two selective advantages that are realized in the deleted form. The mere fact that the genome is 2.8 kb shorter than fd-tet, should speed up the replication. More important may be the fact that the deletion most probably partially reconstitutes the ori(−) structure. The ramifications of a markedly more functional ori(−) would substantially enable more effective production of double stranded DNA.

Another possibility may be related to the packaging of phages for secretion. The A-stem/loop structure, situated adjacent to the B-stem/loop, is the packaging-signal of the phage (Van Wezenbeek et al, 1980). It stands to reason that the tet insert may sterically hinder the packaging of the recombinant phages, a burden that would be removed in the deleted form.

However, the major phenomenon encountered here is the fact that the deletion is detectable only in the F⁺ bacteria. This might be due to the fact that even rare events can be markedly amplified when super infection of bacteria can take place. Obviously, for such super infection, the bacteria must be compatible, a situation only satisfied in F⁺ bacteria. Therefore, it is proposed that the deletion leads to shorter and more efficient replicating forms of the phage, albeit a rare event. Subsequently, however, such deleted phages can be markedly amplified in F⁺ bacteria via super infection thus enabling accumulation of very substantial amounts of the deleted form. However, the possibility cannot be excluded that unknown proteins encoded by the F-episome might contribute to the deletion events either by directly enhancing the process or indirectly inducing the expression of bacterial proteins participating in the process.

In view of the above considerations, it was concluded that the intergenic region of the phage is not the optimal site for introduction of a second pVIII gene. Not that this has not led to an effective “type 88” vector in the past such as the f88.4 vector constructed by Smith (Zhong et al, 1994). In the f88.4 vector, the recombinant pVIII gene was introduced into the tet fragment of fd-tet. However, one would expect with such a vector the gradual appearance of deleted forms of the phages and loss of their peptide inserts displayed by the chimeric pVIII protein. Indeed, such “contaminating”, fast-growing, tetracycline sensitive phages have been reported by Scott when they used the f88.4 vector (Bonnycastle et al, 1996).

An alternative cloning site for the second pVIII gene was tested, namely the non-coding region between the pVIII and pIII genes. The ftac88 described in this report illustrated that such a construction is not only possible but able to produce workable amounts of recombinant phages. However, once again an unexpected obstacle was encountered as substantial amounts of deletions of the recombinant pVIII were observed. This deletion process also occurred in a recA⁻ background, excluding therefore the possibility of classical homologous recombination. In contrast to the situation of the deletion of the tet insert however, the deletion of the modified pVIII gene entails reasonably long direct repeats. Thus, whereas homologous recombination is not an option, the slippage mechanism which is recA independent, described by Feschenko and Lovett (Feschenko et al, 1998), might be responsible for the selective loss of the second pVIII gene in ftac88. This would entail the slipped misalignment of the nascent strand during DNA replication. During replication of the repeated DNA the nascent strand becomes displaced from its template and pairs with the other copy on the template strand. Then, replication is resumed and the slipped misalignment can lead to either deletion or expansion within tandem repeat arrays. Therefore, deletions due to the slippage mechanism are homology dependent, a requirement satisfied in ftac88.

The selective loss of the recombinant pVIII gene further supports the slippage mechanism. This mechanism requires the stabilization of the slipped misalignment structure (Feschenko et al, 1998; Lovett et al, 1996). The longer the displaced nascent strand, the more stabilized the slipped misalignment structure becomes. The recombinant pVIII gene contains a foreign sequence in its 5′ region. This severs the repeat into two parts, a short sequence upstream and a long downstream sequence. The long sequence, therefore, enables the stabilization of the slipped misaligned structure more efficiently than the short one. Therefore, excision sites in the long downstream identical sequence should predominate thus eliminating upstream sequences including the foreign DNA insert.

The actual deletion event may be a very rare one, typical of the slippage mechanism. One can postulate that deleted DNA products could be amplified due to F⁺ dependent superinfection (see discussion above).

Therefore, the last aspect that must be considered is why the pVIIISTS form is stable as opposed to those constructs in which the stuffer containing the trpA terminator is exchanged for a peptide coding-sequence (such as the GV4H3 epitope). The stability rendered by the presence of the trpA transcriptional terminator indicates that transcription might be a positive and necessary factor. The trpA terminator stops the transcription of the recombinant pVIII gene prematurely and consequently no deletion is observed. Transcription through the second homologous DNA repeat appears to be a necessary pre-requisite that provides the opportunity to form the misaligned structure that leads to subsequent deletion during DNA replication. Such a mechanism may explain how misalignment can exist between the first and second repeats at the moment when the first repeat is being replicated yet the second downstream repeat is still in the closed double stranded DNA configuration. In order to mispair the nascent strand to the closed double-stranded repeat, the latter would have to be opened. In the absence of RecA protein, which catalyzes such reactions (West, 1992), transcription might be an efficient mode for opening double stranded DNA. However, as opening double stranded DNA during transcription is transient, this mode would be efficient only when the DNA repeats are in reasonably close proximity. Indeed, the deletion frequency, reported for the slippage mechanism, is correlated directly with the distance separating the DNA repeats (Lovett et al, 1994; Bi et al, 1994). Moreover, this mechanism would require that the wild type pVIII gene be the first to be replicated. Indeed, the most extensive replication during infection is that of the + strand, proceeding from the intergenic region through the wild type pVIII gene to the recombinant pVIII gene (Horiuchi et al, 1976).

As a result of the above analyses and the experiences gained through the construction and characterization of the ftac88 vector, an improved novel vector that corrects for the problems encountered was ultimately designed. This vector has been designated fth1 and has the following advantages.
1. The tetracycline resistance and the recombinant pVIII genes were introduced between the wild type pVIII and pIII genes.
2. To avoid the presence of repetitive sequences of any kind, the two transcription units of the phage genes were separated by inserting a novel HP-terminator. Furthermore, the wild type pVIII C-terminal domain encoding region was reconstituted using alternative codon usage. The recombinant pVIII gene was generated using alternative codon usage as well. These attributes render the fth1 vector genetically stable.
3. The fth1 vector contains the wild type intergenic region. Thus, the minus-strand DNA synthesis occurs as efficiently as in wild type fd rendering fth1 a high copy number vector. This enables one to isolate and purify consistently large amounts of the double-stranded DNA from fth1 harboring bacteria (500 μg per liter as oppose to 50 μg of ftac88). Furthermore, fth1 produces high phage titers −10¹² phages per ml as oppose to 10¹⁰ phages per ml typical for fd-tet and its derivatives.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Bi et al, “recA-independent and recA-dependent intramolecular plasmid recombination”, J Mol Biol 235:414-423 (1994)
Bluthner et al, “Mapping of epitopes recognized by PM/Sc1 autoantibodies with gene-fragment phage display libraries”, J Immunol Methods 198:187-198 (1996)
Bonnycastle et al, “Probing the basis of antibody reactivity with a panel of constrained peptide libraries displayed by filamentous phage”, J Mol Biol 258:747-762 (1996)
Clark A J, “Recombination deficient mutants of Escherichia coli and other bacteria”, Ann Rev Genet 7:67-86 (1973)
Cortese et al, “Epitope discovery using peptide libraries displayed on phage”, Trends Biotechnol 12:262-267 (1994)
Cortese et al, “Identification of biologically active peptides using random libraries displayed on phage”, Curr Opin Biotechnol 6:73-80 (1995)
Cortese et al, “Selection of biologically active peptides by phage display of random peptide libraries. Curr Opin Biotechnol 7:616-621 (1996)
Cwirla et al, “Peptides on phage: a vast library of peptides for identifying ligands”, Proc Natl Acad Sci USA 87:6378-6382 (1990)
Denisova et al, “Humoral immune response to immunocomplexed HIV envelop glycoprotein 120”, AIDS Res Hum Retroviruses 12:901-909 (1996)
Dotto et al, “The functional origin of bacteriophage fl DNA replication: Its Signal and domains”, J Mol Biol 172:507-521 (1984)
Dower W J, “Phage power”, Curr Biol 2:251-253 (1992)
Felici et al, “Selection of antibody ligands from a large library of oligopeptides expressed on a multivalent exposition vector”, J Mol Biol 222:301-310 (1991)
Feschenko et al, “Slipped misalignment mechanisms of deletion formation: analysis of deletion endpoints”, J Mol Biol 276:559-569 (1998)
Goldsmith et al, “Adsorption protein of bacteriophage fd: Isolation, molecular properties, and location in virus”, Biochemistry 16:2686-2694 (1977)
Gray et al “Adsorption complex of filamentous fd virus”, J Mol Biol 146:621-627 (1981)
Greenwood et al, “Multiple display of foreign peptides on a filamentous bacteriophage”, J Mol Biol 220:821-827 (1991)
Hoess et al, “Identification of a peptide which binds to the carbohydrate-specific monoclonal antibody B3”, Gene 128:43-49 (1993)
Holliger et al, “A conserved infection pathway for filamentous bacteriophages is suggested by the structure of the membrane penetration domain of the minor coat protein g3p from phage fd”, Structure 5:265-275 (1997)
Horiuchi et al, “Origin and direction of synthesis of bacteriophage f1 DNA”, Proc Natl Acad Sci USA 73:2341-2345 (1976)
Horton et al, “Gene splicing by overlap extension: tailor-made genes using the polymerase chain reaction”, Biotechniques 8:528-535 (1990)
Iannolo et al, “Modifying filamentous phage capsid: limits in the size of the major capsid protein”, J Mol Biol 248:835-844 (1995)
Kay et al, “An M13 phage library displaying 38-amino-acid peptides as a source of novel sequences with affinity to selected targets”, Gene 128:59-65 (1993)
Kim et al, “Viable deletions of the M13 complementary strand origin”, Proc Natl Acad Sci USA 78:6784-6788 (1981)
Koivunen et al, “Phage libraries displaying cyclic peptides with different ring sizes: ligand specificities of the RGD-directed integrins”, Biotechnology 13:265-270 (1995)
Krebber et al, “Co-selection of cognate-antigen pairs by selectively-infective phages”, FEBS Lett 377:227-231 (1995)
Lane et al, “Epitope mapping using bacteriophage peptide libraries”, Curr Opin Immunol 5:268-271 (1993)
Lovett et al, “Recombination between repeats in Escherichia coli by a recA-independent, proximity-sensitive mechanism”, Mol Gen Genet 245:294-300 (1994)
Lovett et al, “Stabilization of diverged tandem repeats by mismatch repair: evidence for deletion formation via a misaligned replication intermediate”, Proc Natl Acad Sci USA 93:7120-7124 (1996)
Luzzago et al, “Mimicking of discontinuous epitopes by phage displayed peptides, I. Epitope mapping of human H ferritin using a phage library of constrained peptides”, Gene 128:51-57 (1993)
Marvin et al, “Molecular model and structural comparisons of native and mutant class I filamentous bacteriophages Ff (fd, f1, M13), If1 and IKe”, J Mol Biol 235:260-286 (1994)
Matfield et al, “Rec-dependent and Rec-independent recombination of plasmid-borne duplications in Escherichia coli K12”, Mol Gen Genet 199:518-523 (1985)
Matthews et al, “Substrate phage: selection of protease substrates by monovalent phage display”, Science 260:1113-1116 (1993)
McCafferty et al, “Phage antibodies: filamentous phage displaying antibody variable domains”, Nature 348:552-554 (1990)
McCafferty et al, “Phage enzymes: expression and affinity chromatography of functional alkaline phosphatase on the surface of bacteriophage”, Protein Eng 4:955-961 (1992)
Messing J, “New M13 vectors for cloning”, Methods Enzymol 101:20-78 (1983)
Model et al, “Filamentous Bacteriophage”, in The Bacteriophages, Calendar R (ed.), Plenum Press, New York and London, Vol. 2, p. 375 (1988)
Moses et al, “Restructuring the bacteriophage f1 genome: Expression of gene VIII in the intergenic space”, Virology 104:267-278 (1980)
Nohno et al, “Cloning and complete sequence of the Escherichia coli glutamine permease operon (glnHPQ)”, Mol Gen Genet 205:260-269 (1986)
Parmley et al, “Antibody-selectable filamentous fd phage vectors: affinity purification of target genes”, Gene 73:305-318 (1988)
Rasqualini et al, “Organ targeting in vivo using phage display peptide libraries”, Nature 380:364-366 (1996)
Sambrook et al, Molecular Cloning: a Laboratory Manual, Cold Spring Harbor Laboratory Press, Cold Spring Harbor, N.Y. (1989)
Schmitz et al, “Catalytic specificity of phosphotyrosine kinases Blk, Lyn, c-Src and Syk as assessed by phage display”, J Mol Biol 260:664-677 (1996)
Scott et al, “Searching for peptide ligands with an epitope library”, Science 249:386-390 (1990)
Smith G P, “Filamentous phage assembly: morphogenetically defective mutants that do not kill the host”, Virology 167:156-165 (1988)
Smith G P “Surface display and peptide libraries”, Gene 128:1-2 (1993)
Sparks et al, “Identification and characterization of Src SH3 ligands from phage-displayed random peptide libraries”, J Biol Chem 269:23853-23856 (1994)
Stern et al, “Construction and use of a 20-mer phage display epitope library”, in Methods in Molecular Biology: Combinatorial Peptide Library Protocols, S. Cabilly (ed.), Humana Press, Totowa, N.J., p. 137 (1998)
Sugimoto et al, “Studies on bacteriophage fd DNA. IV. The sequence of messenger RNA for the major coat protein gene”, J Mol Biol 111:487-507 (1977)
Van Wezenbeek et al, “Nucleotide sequence of the filamentous bacteriophage M13 DNA genome: comparison with phage fd”, Gene 11:129-148 (1980)
Vieira et al, “Production of single-stranded plasmid DNA”, Methods Enzymol 153:3-11 (1987)
West S C, “Enzymes and molecular mechanisms of genetic recombination”, Annu Rev Biochem 61:603-640 (1992)
Willis et al, “Immunological properties of foreign peptides in multiple display on a filamentous bacteriophage”, Gene 128:79-83 (1993)
Wrighton et al, “Small peptides as potent mimetics of the protein hormone erythropoietin”, Science 273:458-463 (1996)
Zacher et al, “A new filamentous phage cloning vector: fd-tet”, Gene 9:127-140 (1980)
Zhong et al, “Conformational mimicry of a chlamydial neutralization epitope on filamentous phage”, J Biol Chem 269:24183-24188 (1994

1. A DNA molecule comprising the DNA of a filamentous phage into which has been inserted a recombinant DNA sequence comprising a multiple cloning site comprising a series of restriction sites that do not otherwise appear in said DNA of the filamentous phage, said recombinant DNA sequence being designed and inserted such that the multiple cloning site is placed between a terminator for the wild type pVIII gene and an initiator for the wild type pIII gene.

2. A DNA molecule in accordance with claim 1, wherein a positive selection marker is present in the multiple cloning site.

3. A DNA molecule in accordance with claim 1, wherein an additional bacteriophage gene is present in the multiple cloning site.

4. A DNA molecule in accordance with claim 1, wherein said additional bacteriophage gene is a second pVIII gene.

5. A DNA molecule in accordance with claim 1, wherein said additional bacteriophage gene is a second pIII gene.

6. A DNA molecule in accordance with claim 1, wherein said recombinant DNA sequence is designed and inserted such that a DNA sequence encoding a foreign peptide is incorporated into the pIII gene at the N-terminal region thereof.

7. A DNA molecule in accordance with claim 1, wherein said multiple cloning site placed between said wild type pVIII gene (the first pVIII gene) and the wild type pIII gene comprises DNA encoding an additional pVIII protein (the second pVIII gene) and DNA encoding a polypeptide of interest other than pVIII.

8. A DNA molecule in accordance with claim 7, wherein the DNA encoding the second pVIII gene uses alternative codons which are other than the native codons for encoding one or more of the amino acid residues of the pVIII protein, such that no stretch of more than 30 bases of the native sequence appear therein.

9. A DNA molecule in accordance with claim 8, wherein no stretch of more than 20 bases of the native sequence appear in the DNA encoding the second pVIII gene.

10. A DNA molecule in accordance with claim 7, wherein the native pVIII DNA sequence is separated from the recombinant pVIII DNA sequence by a terminator which is not native to the filamentous phage DNA.

11. A DNA molecule in accordance with claim 7, wherein DNA encoding the native amino acid sequence of the pVIII C-terminal/pIII −35 box is maintained at the region immediately upstream to the initiation of the pIII gene.

12. A DNA molecule in accordance with claim 11, wherein said region encoding the pVIII C-terminal domain in said C-terminal/−35 box uses alternative codons which are other than the native codons, such that no stretch of more than 30 bases of the native sequence appear therein.

13. A DNA molecule in accordance with claim 12, wherein no stretch of more than 20 bases of the native sequence appear in the region encoding the pVIII C-terminal/−35 box.

14. A DNA molecule in accordance with claim 7, wherein a positive selection marker is inserted between the native and recombinant pVIII genes.

15. A DNA molecule in accordance with claim 14, wherein said positive selection marker is the tetracycline resistance gene.

16. A DNA molecule in accordance with claim 15, wherein a unidirectional promoter is substituted for the native bidirectional tetracycline resistance gene promoter.

17. A DNA molecule in accordance with claim 7, wherein the native intergenic space region is present.

18. A DNA molecule in accordance with claim 7, wherein a promoter is used for the second pVIII gene and DNA encoding the peptide of interest, which promoter is not native to the native filamentous phage.

19. A DNA molecule in accordance with claim 7, wherein said recombinant DNA sequence is designed and inserted such that a DNA sequence encoding a second peptide of interest is incorporated into the pIII gene at the N-terminal region thereof.

20. A filamentous phage encoded by the DNA of claim 7.

21. A DNA molecule in accordance with claim 7 wherein said polypeptide of interest is a random peptide.

22. A library of filamentous phages encoded by a DNA molecule in accordance with claim 21, wherein said library comprises a plurality of phages each having a different random peptide as the polypeptide of interest.

23. A library in accordance with claim 22, wherein the polypeptides of interest in said plurality of phages comprise all possible random peptides of a given length or a fraction thereof which maintains the complexity thereof.

24. A filamentous phage encoded by a DNA molecule in accordance with claim 7, wherein said polypeptide of interest is a peptide which comprises a contiguous fragment of the sequence of an antigen of interest.

25. A library of filamentous phages in accordance with claim 24, wherein said library comprises a plurality of phages, said peptide in each of said plurality of phages comprising a different contiguous fragment of the sequence of the antigen of interest.

26. A library in accordance with claim 25, wherein the polypeptides of interest in said plurality of phages comprise all possible overlapping contiguous fragments of the antigen of interest.

27. A method of screening for a molecule which binds to a peptide of interest comprising bringing a molecule to be screened into contact with a filamentous phage in accordance with claim 20 which expresses said peptide of interest and, if said molecule binds to said peptide of interest, identifying and producing said molecule.

28. A method of screening for peptides which bind to a molecule of interest comprising bringing the molecule of interest into contact with a library of filamentous phages in accordance with claim 22; identifying any peptide expressed by a phage which binds to said molecule; and producing a polypeptide which comprises said peptide.

29. A method of screening for peptides which bind to a molecule of interest comprising bringing the molecule of interest into contact with a library of filamentous phages in accordance with claim 25; identifying any peptide expressed by a phage which binds to said molecule; and producing a polypeptide which comprises said peptide.

30. A DNA molecule comprising the DNA of a filamentous phage, into which has been inserted between the wild type pVIII gene (the first pVIII gene) and the wild type pIII gene, recombinant DNA sequence encoding an additional pVIII protein (the second pVIII gene) and DNA containing a region of bases flanked by two identical restriction enzyme sites, which sites do not appear anywhere else in the DNA molecule.

31. A DNA construct for insertion into the DNA of a filamentous phage, said construct comprising a multiple cloning site comprising a series of restriction sites that do not otherwise appear in the DNA of the phage into which the construct is intended to be inserted, said construct having one end compatible for ligation to a restriction enzyme site upstream of the pIII gene of the phage into which the construct is intended to be inserted and another end compatible for ligation to a restriction enzyme site downstream of the pVIII gene of the phage into which the construct is intended to be inserted.

32. A DNA construct in accordance with claim 31 further including a positive selection marker.

33. A DNA construct in accordance with claim 31 further including a second pVIII gene.
